(19) United States
(12) Patent Application Publication
(10) Pub. No.: US 2026/0062365 A1
(43) Pub. Date: Mar. 5, 2026

(54) METHOD FOR PRODUCING trans-1,2-DIFLUOROETHYLENE (HFO-1132E)

(71) Applicant: Honeywell International Inc., Charlotte, NC (US)

(72) Inventors: Haiyou Wang, Amherst, NY (US); Akbar Mahdavi-Shakib, Buffalo, NY (US)

(21) Appl. No.: 19/306,508

(22) Filed: Aug. 21, 2025

Related U.S. Application Data
(60) Provisional application No. 63/688,702, filed on Aug. 29, 2024.
(60) Provisional application No. 63/746,757, filed on Jan. 17, 2025.

(51) Int. Cl. C07C 17/25; C07C 19/14

(52) U.S. Cl. CPC C07C 17/25; C07C 19/14; C07C 2523/44

(57) ABSTRACT

Production of HFO-1132 and, in particular, HFO-1132E, is produced from chlorotrifluoroethylene (CTFE) and/or trifluoroethylene (HFO-1123). In a first step, 1,1,2-trifluoroethane (HFC-143) is produced by hydrogenating chlorotrifluoroethylene (CTFE) and/or trifluoroethylene (HFO-1123) by reaction with hydrogen in the presence of a catalyst at a temperature of between about 75° C. and about 225° C. The 1,1,2-trifluoroethane (HFC-143) may then be dehydrohalogenated in the presence of a catalyst to produce trans-1,2-difluoroethylene (HFO-1132E) and cis-1,2-difluoroethylene (HFO-1132Z). The cis-1,2-difluoroethylene (HFO-1132Z) may then be isomerized to produce trans-1,2-difluoroethylene (HFO-1132E).

### CROSS-REFERENCE TO RELATED APPLICATIONS

This patent application claims the benefit under 35 U.S.C. § 119(e) of U.S. Provisional Patent Application Ser. No. 63/688,702, filed on Aug. 29, 2024, and U.S. Provisional Patent Application Ser. No. 63/746,757, filed on Jan. 17, 2025, the disclosures of each of which are incorporated by reference herein in their entireties.

### FIELD

The present disclosure is directed to a method for producing trans-1,2-difluoroethylene (HFO-1132E) from chlorotrifluoroethylene (CTFE or CFC-1113) and/or trifluoroethylene (TrFE or HFO-1123).

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Improved methods for the production of HFO-1132 and, in particular, HFO-1132E, are desired.

### SUMMARY

The present disclosure is based on the discovery that HFO-1132 and, in particular, HFO-1132E, is produced from chlorotrifluoroethylene (CTFE) and/or trifluoroethylene (HFO-1123) via catalytic reaction steps in which desirable intermediates are produced in controlled amounts while production of undesired byproducts is minimized.

In a first step, 1,1,2-trifluoroethane (HFC-143) is produced by hydrogenating chlorotrifluoroethylene (CTFE) and/or trifluoroethylene (HFO-1123) by reaction with hydrogen in the presence of a catalyst during which desirable intermediates are produced in controlled amounts while production of undesired byproducts is minimized. The 1,1,2-trifluoroethane (HFC-143) is then dehydrohalogenated in the presence of a catalyst to produce trans-1,2-difluoroethylene (HFO-1132E) and/or cis-1,2-difluoroethylene (HFO-1132Z). The cis-1,2-difluoroethylene (HFO-1132Z) is then isomerized to produce trans-1,2-difluoroethylene (HFO-1132E).

The overall reaction methods and/or specific reaction conditions of the first step for producing the desired product 1,1,2-trifluoroethane (HFC-143) from chlorotrifluoroethylene (CTFE) and/or trifluoroethylene (HFO-1123) is selectively tailored, such as with catalyst selection and/or conditioning, to advantageously convert desirable intermediates to the desired product 1,1,2-trifluoroethane (HFC-143) and/or minimize the formation of undesired byproducts.

In one form thereof, the present disclosure provides a method for producing 1,1,2-trifluoroethane (HFC-143), comprising: hydrogenating a feed material (e.g., reactant composition) comprising chlorotrifluoroethylene (CTFE) and/or trifluoroethylene (HFO-1123) by reaction with hydrogen in the presence of a catalyst at a temperature of between about 75° C. and about 225° C. to produce a product mixture comprising 1,1,2-trifluoroethane (HFC-143). The hydrogenation step is selectively tailored, such as with catalyst selection and/or conditioning, to maximize the production of desired product 1,1,2-trifluoroethane (HFC-143) and desired intermediates including 1-chloro-1,1,2-trifluoroethane (HCFC-133b), 1-chloro-1,2,2-trifluoroethane (HCFC-133), and trifluoroethylene (HFO-1123), while minimizing the production of undesired byproducts including ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,1-trifluoroethane (HFC-143a), etc.

In another form thereof, the present disclosure provides a method for producing trans-1,2-difluoroethylene (HFO-1132E), comprising dehydrohalogenating 1,1,2-trifluoroethane (HFC-143) in the presence of a catalyst to produce trans-1,2-difluoroethylene (HFO-1132E) and cis-1,2-difluoroethylene (HFO-1132Z); and isomerizing cis-1,2-difluoroethylene (HFO-1132Z) to produce trans-1,2-difluoroethylene (HFO-1132E).

### BRIEF DESCRIPTION OF THE DRAWINGS

FIG. 1 is a schematic diagram of the apparatus used in Examples 1˜4 for the conversion of chlorotrifluoroethylene (CTFE) and/or trifluoroethylene (HFO-1123) to 1,1,2-trifluoroethane (HFC-143).

FIG. 2 is a schematic reactor configuration corresponding to Examples 7-50.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, “CTFE” and “CFC-1113” are used interchangeably to refer to chlorotrifluoroethylene.

As used herein, “HFO-1123” and “TrFE” are used interchangeably to refer to trifluoroethylene.

As used in the Examples section, the designation “R” is used in connection with the various fluorine-containing molecules described herein, for example, “R-143” refers to 1,1,2-trifluoroethane (HFC-143).

As used herein, the phrase “desired product” is 1,1,2-trifluoroethane (HFC-143).

As used herein, the phrase “desired intermediates” include one or more of 1-chloro-1,1,2-trifluoroethane (HCFC-133b), 1-chloro-1,2,2-trifluoroethane (HCFC-133), and trifluoroethylene (HFO-1123).

As used herein, the phrase “undesired byproducts” include one or more of ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,1-trifluoroethane (HFC-143a).

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation when substantially pure reactant is used:

$$\% conversion of molecule X = ( 100 - molecule X mol . \% in the organic components of a product mixture )$$

When the reactant includes impurities or recycled components, i.e., is a component in a reaction mixture, the conversion of a reactant molecule (molecule X) during a reaction is calculated using the following equations:

$$\% conversion of molecule X = ( change in X mol . \% ) / ( X mol \% in the reactant mixture )$$

$$OR$$

$$\% conversion of molecule X = ( X mol \% in the reactant mixture - X mol \% in the product mixture ) / ( X mol \% in the reactant mixture )$$

As used herein, selectivity to a molecule formed during a reaction (molecule X) is calculated using the following equation:

$$\% selectivity to molecule X = mol . \% of molecule X in the organic components of a product mixture / ( 100 - mol . \% of reactant molecules in the organic components of a product mixture ) \times 100.$$

When the substrate conversion rate is 100%, the mole percentages of each molecule in the resulting product mixture are equal to the selectivity of each molecule.

### II. Overview

The present disclosure provides a method for producing E-1,2-difluoroethylene (HFO-1132E) from chlorotrifluoroethylene (CTFE) and/or trifluoroethylene (HFO-1123) according to a three-step process shown below (“Process 1”), which includes the following three steps: (i) hydrogenating chlorotrifluoroethylene (CTFE) and/or trifluoroethylene (HFO-1123) to produce 1,1,2-trifluoroethane (HFC-143), (ii) dehydrohalogenating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E). The hydrogenation step is selectively tailored, such as with catalyst selection and/or conditioning, to maximize the production of desired product and intermediates while minimizing the production of undesired byproducts. The dehydrohalogenation step (step (ii)) may be conducted in a liquid phase or a vapor phase reactor.

Schematic equations for the three steps of Process 1 are represented below:

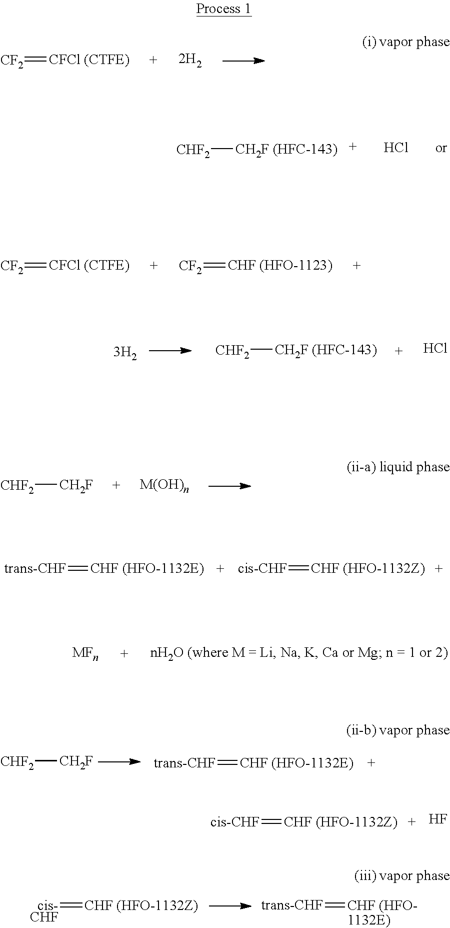

Without wishing to be bound by theory, Step (i) may proceed through an intermediate of trifluoroethylene (HFO-1123), wherein a feed material (e.g., reactant composition) including chlorotrifluoroethylene (CTFE) is first hydrogenated to produce the trifluoroethylene (HFO-1123) as an intermediate, which intermediate is itself then hydrogenated to produce CHF₂—CH₂F (HFC-143). Alternatively, both CTFE and HFO-1123 are used as feed materials in Step (i) to be hydrogenated to produce HFC-143.

Moreover, without wishing to be bound by theory, Step (i) may include a number of non-limiting possible side reactions, including but not limited to:

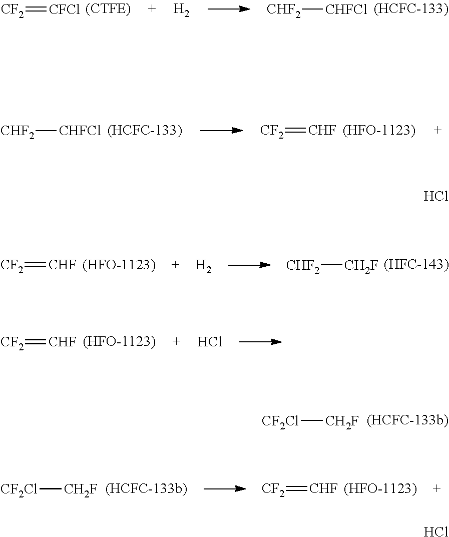

Further details regarding each of Steps (i), (ii), (iii) are set forth below.

### III. Step (i)

### General Process

The hydrogenation reaction of Step (i) may be carried out in the vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Products may be flowed through one or more scrubbers (e.g., a KOH scrubber, an NaOH scrubber) to remove byproducts from the reaction, such as hydrogen fluoride (HF) and/or hydrogen chloride (HCl), and the reaction products may be collected by capture in a cooled cylinder, for example.

An organic feedstock (e.g., feed material or reactant composition) which comprises the starting material (e.g., CF₂═CFCl (CTFE) and/or trifluoroethylene (HFO-1123)), a supply of inert carrier gas, and a hydrogenation gas are fed into a reactor under the reaction conditions as described herein. The reaction may be monitored by taking samples to conduct a GC analysis.

### Catalysts

The catalyst and process conditions play an important role in the reaction. Specifically, in the hydrogenation reaction of Step (i), the catalyst may comprise a metal such as palladium (Pd), platinum (Pt), rhodium (Rh), iridium (Ir), nickel (Ni), cobalt (Co), iron (Fe), ruthenium (Ru), or a combination thereof. The metal catalyst used to catalyze the reaction may be palladium, platinum, or a combination of palladium and platinum.

### Catalyst Support

The catalyst active to catalyze the hydrogenation reaction of Step (i) includes a metal catalyst on a support. Examples of common catalyst supports include activated carbon, porous aluminosilicate typified by zeolite, aluminum oxide (alumina), silicon oxide, titanium oxide, zirconium oxide, zinc oxide, aluminum fluoride, and the like.

Alumina exists in several metastable crystalline structures (e.g., eta(η)-, gamma(γ)-, delta(δ)-, theta(θ)-, beta(β)-, kappa(κ)-, chi(χ), and alpha(α)-alumina). When those that are not so stable (e.g., gamma(γ)-alumina) are chosen and used for hydrogenation reaction of fluorine-containing compounds (e.g., CTFE, HFO-1132, etc.), undesired reactions may take place between the support and the organic compound resulting in undesired byproducts to be formed and the loss or reduction of catalyst activity. Hence, there is a need for means by which such undesired reactions can be reduced or avoided.

In the hydrogenation reaction of Step (i), the catalysts described herein is supported on activated carbon or an inert form of alumina, for example, an alumina support including alpha(α) alumina, theta(θ) alumina, delta(δ) alumina, or a mixture thereof.

In the hydrogenation reaction of Step (i), the supported catalyst may be produced by impregnation of any of the suitable supports with a solution of a compound of the desired metal constituent. The support may also be in the form of pellets. The size of the pellets may be as little as about 1/32 inch, about 1/16 inch, or as great as about ⅛ inch, about 3/16 inch, about ¼ inch, about 5/16 inch, about ⅜ inch, about 7/16 inch, or about ½ inch, or within any range encompassing any two of these values as endpoints, for example, from about 1/32 inch to about ½ inch, preferably from about 1/16 inch to about 3/16 inch, or more preferably about ⅛ inch. After the impregnation step, the solvent (e.g., water) may be removed using heat or under vacuum resulting in a solid mass which can be further dried and reduced to form active metal catalyst.

In the hydrogenation reaction of Step (i), the metal catalysts supported on various catalyst supports are listed in Table 1 below.

TABLE 1
Supported Metal Catalysts - Step (i) Hydrogenation Reaction

| Catalyst | Support |
|---|---|
| Pd | alpha(α)-Al₂O₃ |
| Pt | alpha(α)-Al₂O₃ |
| Rh | alpha(α)-Al₂O₃ |
| Ru | alpha(α)-Al₂O₃ |
| Ir | alpha(α)-Al₂O₃ |
| Fe | alpha(α)-Al₂O₃ |
| Co | alpha(α)-Al₂O₃ |
| Ni | alpha(α)-Al₂O₃ |
| Pd | theta(θ)-Al₂O₃ |
| Pt | theta(θ)-Al₂O₃ |
| Rh | theta(θ)-Al₂O₃ |
| Ru | theta(θ)-Al₂O₃ |
| Ir | theta(θ)-Al₂O₃ |
| Fe | theta(θ)-Al₂O₃ |
| Co | theta(θ)-Al₂O₃ |
| Ni | theta(θ)-Al₂O₃ |
| Pd | delta(δ)-Al₂O₃ |
| Pt | delta(δ)-Al₂O₃ |
| Rh | delta(δ)-Al₂O₃ |
| Ru | delta(δ)-Al₂O₃ |
| Ir | delta(δ)-Al₂O₃ |
| Fe | delta(δ)-Al₂O₃ |
| Co | delta(δ)-Al₂O₃ |
| Ni | delta(δ)-Al₂O₃ |
| Pd | Activated carbon |
| Pt | Activated carbon |
| Rh | Activated carbon |
| Ru | Activated carbon |
| Ir | Activated carbon |
| Fe | Activated carbon |
| Co | Activated carbon |
| Ni | Activated carbon |

### Catalyst Loadings

When a metal catalyst such as Pd, Pt, Rh, Ru, or Ir is used, its loading on a catalyst support (e.g., alpha(α)-Al₂O₃, or theta(θ)-Al₂O₃, or delta(δ)-Al₂O₃, or activated carbon) is as little as about 0.01 wt. %, about 0.05%, about 0.1 wt. %, about 0.15%, about 0.2 wt. %, about 0.25%, about 0.3 wt. %, about 0.4 wt. %, or as great as about 0.5 wt. %, about 1 wt. %, about 2 wt. %, about 3 wt. %, about 5 wt. %, or about 10 wt. %, based on a total weight of the catalyst and support, or within any range encompassing any two of these values as endpoints, for example, from about 0.05 wt. % to about 2 wt. %, preferably from about 0.1 wt. % to about 1 wt. %, or more preferably from about 0.2 wt. % to about 0.5 wt. %. Specific examples of additional suitable ranges are set forth below in Table 2. The numerical ranges set forth in Table 2 below are understood to be prefaced by “about”.

TABLE 2
Loading for Catalysts (Pd, Pt, Rh, Ru, or Ir) on Supports
(alpha(α)-Al₂O₃, theta(θ)-Al₂O₃, or delta(δ)-Al₂O₃
or activated carbon) - Each Catalyst/Support Combination
(Each Row) in Table 1 - Step (i) Hydrogenation Reaction

| From (wt. %) | To (wt. %) |
|---|---|
| 0.01 | 10 |
| 0.01 | 5 |
| 0.01 | 3 |
| 0.01 | 2 |
| 0.01 | 1 |
| 0.01 | 0.5 |
| 0.01 | 0.4 |
| 0.01 | 0.3 |
| 0.01 | 0.2 |
| 0.01 | 0.1 |
| 0.05 | 10 |
| 0.05 | 5 |
| 0.05 | 3 |
| 0.05 | 2 |
| 0.05 | 1 |
| 0.05 | 0.5 |
| 0.05 | 0.4 |
| 0.05 | 0.3 |
| 0.05 | 0.2 |
| 0.05 | 0.1 |
| 0.1 | 10 |
| 0.1 | 5 |
| 0.1 | 1 |
| 0.1 | 0.5 |
| 0.1 | 0.3 |
| 0.1 | 0.2 |
| 0.2 | 10 |
| 0.2 | 5 |
| 0.2 | 1 |
| 0.2 | 0.5 |
| 0.2 | 0.3 |

When a metal catalyst such as Fe, Co, Ni is used, its loading on a catalyst support (e.g., alpha(α)-Al₂O₃, or theta(θ)-Al₂O₃, or delta(δ)-Al₂O₃) is as little as about 5 wt. %, about 10 wt. %, about 20 wt. %, or about 30 wt. %, about 40 wt. %, or as great as about 50 wt. %, about 60 wt. %, about 70 wt. %, or about 80 wt. %, based on a total weight of the catalyst and support, or within any range encompassing any two of these values as endpoints, for example, from about 5 wt. % to about 80 wt. %, preferably from about 10 wt. % to about 70 wt. %, or more preferably from about 20 wt. % to about 30 wt. %. Specific examples of additional suitable ranges are set forth below in Table 3. The numerical ranges set forth in Table 3 below are understood to be prefaced by “about”.

TABLE 3
Loading for Catalysts (Fe, Co, or Ni) on Supports (alpha(α)-
Al₂O₃, theta(θ)-Al₂O₃, or delta(δ)-Al₂O₃ or activated
carbon) - Each Catalyst/Support Combination (Each Row)
in Table 1 - Step (i) Hydrogenation Reaction

| From (wt. %) | To (wt. %) |
|---|---|
| 5 | 80 |
| 5 | 70 |
| 5 | 60 |
| 5 | 50 |
| 5 | 40 |
| 5 | 30 |
| 5 | 20 |
| 10 | 80 |
| 10 | 70 |
| 10 | 60 |
| 10 | 50 |
| 10 | 40 |
| 10 | 30 |
| 10 | 20 |
| 20 | 80 |
| 30 | 80 |
| 40 | 80 |
| 50 | 80 |
| 60 | 80 |
| 70 | 80 |
| 20 | 30 |
| 30 | 40 |
| 40 | 50 |
| 50 | 60 |
| 60 | 70 |

When a palladium catalyst is used, the loading of palladium on a catalyst support (e.g., alpha(α)-Al₂O₃, or theta(θ)-Al₂O₃, or delta(δ)-Al₂O₃) is from about 0.01 wt. % to about 5 wt. %, preferably from about 0.05 wt. % to about 2 wt. %, and more preferably from about 0.1 wt. % to about 1 wt. %.

### Catalyst BET Surface Area

The BET (Brunauer, Emmet, and Teller) analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

For each catalyst/support combination (each row) in Table 1 used in the hydrogenation reaction of Step (i), the BET surface area may be as low as about 0.1 m²/g, about 1 m²/g, about 3 m²/g, about 5 m²/g, about 10 m²/g, about 15 m²/g, about 20 m²/g 2, about 30 m²/g, about 40 m²/g, about 50 m²/g, about 100 m²/g, or about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, or within any range encompassed by any of the foregoing values as endpoints, for example, from about 0.1 m²/g to about 500 m²/g, about 1 m²/g to about 500 m²/g, about 1 m²/g to about 400 m²/g, preferably from about 1 m²/g to about 300 m²/g, or more preferably from about 1 m²/g to about 200 m²/g.

For carbon supported metal catalysts (Pd, Pt, Rh, Ru, Ir, Fe, Co, or Ni) used in the hydrogenation reaction of Step (i), the BET surface area may be from about 100 m²/g to about 3000 m²/g, preferably from about 200 m²/g to about 2000 m²/g, more preferably from about 500 m²/g to about 1500 m²/g, and most preferably from about 1000 m²/g to about 1500 m²/g. Specific examples of additional suitable ranges are set forth below in Table 4a.

For alumina (alpha(α)-Al₂O₃, theta(θ)-Al₂O₃, or delta(δ)-Al₂O₃) supported metal catalysts (Pd, Pt, Rh, Ru, Ir, Fe, Co, or Ni) used in the hydrogenation reaction of Step (i), the BET surface area may be from about 0.5 m²/g to about 500 m²/g, preferably from about 1 m²/g to about 200 m²/g, more preferably from about 1 m²/g to about 100 m²/g, and most preferably from about 1 m²/g to about 20 m²/g. Specific examples of additional suitable ranges are set forth below in Table 4b. The numerical ranges set forth in Tables 4a and 4b below are understood to be prefaced by “about”.

TABLE 4a
BET Surface Area of Catalysts (Pd, Pt, Rh, Ru, Ir, Fe, Co, or Ni)
on Support (Activated Carbon) - Step (i) Hydrogenation Reaction

| From (m²/g) | To (m²/g) |
|---|---|
| 100 | 3000 |
| 100 | 2000 |
| 100 | 1500 |
| 200 | 3000 |
| 200 | 2000 |
| 200 | 1500 |
| 500 | 3000 |
| 500 | 2000 |
| 500 | 1500 |
| 1000 | 3000 |
| 1000 | 2000 |
| 1000 | 1500 |
| 100 | 200 |
| 200 | 500 |
| 500 | 1000 |
| 1000 | 1500 |
| 1500 | 2000 |
| 2000 | 3000 |

TABLE 4b
BET Surface Area of Catalysts (Pd, Pt, Rh, Ru,
Ir, Fe, Co, or Ni) on Supports (alpha(α)-Al₂O₃, or
theta(θ)-Al₂O₃, or delta(δ)-Al₂O₃)

| From (m²/g) | To (m²/g) |
|---|---|
| 0.5 | 500 |
| 0.5 | 200 |
| 0.5 | 100 |
| 0.5 | 20 |
| 1 | 500 |
| 1 | 200 |
| 1 | 100 |
| 1 | 20 |
| 0.5 | 1 |
| 1 | 50 |
| 50 | 100 |
| 100 | 200 |
| 200 | 500 |

When a palladium catalyst is used on an alumina support (e.g., alpha(α)-Al₂O₃, or theta(θ)-Al₂O₃, or delta(δ)-Al₂O₃), the BET surface area may be from about 1 m²/g to about 300 m²/g, from about 1 m²/g to about 200 m²/g, preferably from about 1 m²/g to about 100 m²/g, or more preferably from about 1 m²/g to about 20 m²/g.

### Catalyst Pretreatment

For each catalyst/support combination (each row) in Table 1, the catalyst may be pretreated by a variety of methods to improve its performance and effectiveness in the reaction. For example, the catalyst may be dried at elevated temperatures, as low as about 100° C., about 150° C., about 200° C., about 250° C., about 300° C., or as high as about 350° C., about 360° C., about 370° C., about 380° C., about 390° C., about 400° C. or within any range encompassed by two of the foregoing values as endpoints. Specific examples of additional suitable ranges are set forth below in Table 5. The numerical ranges set forth in Table 5 below are understood to be prefaced by “about”.

TABLE 5
Pre-treatment Drying Temperatures for Catalysts (Pd, Pt, Rh, Ru, Ir,
Fe, Co, or Ni) on Supports (alpha(α)-Al₂O₃, theta(θ)-Al₂O₃, or delta(δ)-
Al₂O₃ or activated carbon) - Each Catalyst/Support Combination
(Each Row) in Table 1 - Step (i) Hydrogenation Reaction

| From (° C.) | To (° C.) |
|---|---|
| 100 | 400 |
| 120 | 400 |
| 150 | 400 |
| 200 | 400 |
| 100 | 400 |
| 100 | 350 |
| 100 | 300 |
| 100 | 250 |
| 100 | 200 |
| 100 | 150 |
| 100 | 120 |
| 120 | 200 |
| 200 | 300 |
| 300 | 400 |

For each catalyst/support combination (each row) in Table 1 used in the hydrogenation reaction of Step (i), as part of the catalyst pretreatment, the catalysts (e.g., Pd, Pt, Rh, Ru, Ir, Fe, Co, or Ni) may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints, for example, from about 1 hour to about 2 hours, from about 2 hours to about 3 hours, from about 3 hours to about 4 hours, from about 2 hours to about 5 hours, from about 2 hours to about 10 hours, from about 2 hours to about 20 hours, preferably from about 1 hour to about 4 hours, or more preferably from about 1 hours to about 3 hours.

When a palladium catalyst is used on an alumina support (e.g., alpha(α)-Al₂O₃, or theta(θ)-Al₂O₃, or delta(δ)-Al₂O₃), the catalyst may be dried at a temperature of from about 100° C. to about 700° C., from about 120° C. to about 700° C., from about 200° C. to about 700° C., preferably from about 120° C. to about 500° C., or more preferably from about 120° C. to about 300° C.

When a palladium catalyst is used on an alpha alumina support, the catalyst may be exposed to an inert gas such as N₂ for from about 1 hour to about 20 hours, preferably from about 1 hour to about 10 hours, or more preferably from about 1 hour to about 3 hours.

### Reaction Condition—Temperature

For hydrogenation reactions of Step (i) using each catalyst/support combination (each row) in Table 1, the reaction temperature is as low as about 50° C., about 75° C., about 100° C., about 120° C., about 125° C., about 150° C., about 200° C., or as high as about 225° C., about 250° C., about 300° C., about 325° C., about 350° C. or within any range encompassed by two of the foregoing values as endpoints, such as from about 75° C. to about 250° C., or from about 120° C. to about 200° C., for example. The temperature is preferably from about 75° C. to about 225° C., and more preferably from about 100° C. to about 200° C. Specific examples of additional suitable ranges are set forth below in Table 6. The numerical ranges set forth in Table 6 below are understood to be prefaced by “about”.

TABLE 6
Reaction Temperature when using Catalysts (Pd, Pt, Rh, Ru, Ir,
Fe, Co, or Ni) on Supports (alpha(α)-Al₂O₃, theta(θ)-Al₂O₃, or delta(δ)-
Al₂O₃ or activated carbon) - Each Catalyst/Support Combination
(Each Row) in Table 1 - Step (i) Hydrogenation Reaction

| From (° C.) | To (° C.) |
|---|---|
| 50 | 350 |
| 50 | 325 |
| 50 | 300 |
| 50 | 250 |
| 50 | 225 |
| 50 | 200 |
| 50 | 350 |
| 75 | 325 |
| 75 | 300 |
| 75 | 250 |
| 75 | 225 |
| 75 | 200 |
| 100 | 350 |
| 100 | 325 |
| 100 | 300 |
| 100 | 250 |
| 100 | 225 |
| 100 | 200 |
| 75 | 100 |
| 100 | 120 |
| 120 | 200 |
| 200 | 225 |
| 225 | 250 |
| 250 | 300 |
| 300 | 325 |

When a palladium catalyst is used on an alpha alumina support for a hydrogenation reaction of Step (i), the reaction temperature is from about 75° C. to about 300° C., preferably from about 100° C. to about 250° C., most preferably from about 100° C. to about 200° C.

As demonstrated by the Examples herein, the overall selectivity to CHF2-CH2F (HCFC-143) and its associated recyclable intermediates such as 1-chloro-1,1,2-trifluoroethane (HCFC-133b), 1-chloro-1,2,2-trifluoroethane (HCFC-133), and trifluoroethylene (HFO-1123) may increase with lower temperature because of decreased formation of undesired byproducts such as ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,1-trifluoroethane (HFC-143a), etc.

### Reaction Condition—Catalyst Contact Time

For hydrogenation reactions of Step (i) using each catalyst/support combination (each row) in Table 1, the contact time of the reactants with the catalyst as listed in Table 1 is as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time is preferably from about 1 second to about 60 seconds. Specific examples of additional suitable ranges are set forth below in Table 7. The numerical ranges set forth in Table 7 below are understood to be prefaced by “about”.

When a palladium catalyst is used on an alpha alumina support, the contact time is from about 1 second to about 120 seconds, or from about 1 second to about 60 seconds, preferably from about 5 seconds to about 60 seconds, most preferably from about 10 seconds to about 30 seconds.

TABLE 7
Contact Time of Reactants with Catalysts (Pd, Pt, Rh, Ru, Ir, Fe,
Co, or Ni) on Supports (alpha(α)-Al₂O₃, theta(θ)-Al₂O₃, or delta(δ)-
Al₂O₃ or activated carbon) - Each Catalyst/Support Combination
(Each Row) in Table 1 - Step (i) Hydrogenation Reaction

| From (seconds) | To (seconds) |
|---|---|
| 0.1 | 120 |
| 0.1 | 100 |
| 0.1 | 80 |
| 0.1 | 60 |
| 1 | 120 |
| 1 | 100 |
| 1 | 80 |
| 1 | 60 |
| 0.1 | 1 |
| 1 | 10 |
| 10 | 20 |
| 20 | 30 |
| 30 | 40 |
| 40 | 50 |
| 50 | 60 |
| 60 | 80 |
| 80 | 100 |
| 100 | 120 |

### Reaction Condition—Pressure

For hydrogenation reactions of Step (i) using each catalyst/support combination (each row) in Table 1, the reaction pressure is as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 100 psig. Specific examples of additional suitable ranges are set forth below in Table 8. The numerical ranges set forth in Table 8 below are understood to be prefaced by “about”.

When a palladium catalyst is used on an alpha alumina support, the pressure is from about 1 psig to about 300 psig, preferably from about 1 psig to about 200 psig, most preferably from about 10 psig to about 100 psig.

TABLE 8
Reaction Pressure using Catalysts (Pd, Pt, Rh, Ru, Ir, Fe, Co,
or Ni) on Supports (alpha(α)-Al₂O₃, theta(θ)-Al₂O₃, or delta(δ)-
Al₂O₃ or activated carbon) - Each Catalyst/Support Combination
(Each Row) in Table 1 - Step (i) Hydrogenation Reaction

| From (psig) | To (psig) |
|---|---|
| 1 | 300 |
| 1 | 250 |
| 1 | 200 |
| 1 | 150 |
| 1 | 100 |
| 10 | 300 |
| 10 | 250 |
| 10 | 200 |
| 10 | 150 |
| 10 | 100 |
| 1 | 10 |
| 10 | 50 |
| 50 | 100 |
| 100 | 150 |
| 150 | 200 |
| 200 | 250 |
| 250 | 300 |

### Reaction Conditions—Hydrogen Mole Ratio

For hydrogenation reactions of Step (i) using each catalyst/support combination (each row) in Table 1, the mole ratio of hydrogen to the feed material (e.g., CF₂═CFCl (CTFE) and/or trifluoroethylene (HFO-1123)) may be as little about 1:1, about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to feed material may be preferably from about 2:1 to about 15:1, and more preferably from about 3:1 to about 10:1.

When a palladium catalyst is used on an alpha alumina support, the mole ratio of hydrogen to the feed material (e.g., CF₂═CFCl (CTFE) and/or trifluoroethylene (HFO-1123)) may be from about 1:1 to about 20:1, preferably from about 2:1 to about 15:1, more preferably from about 3:1 to about 5:1, and most preferably about 4:1.

A summary of the preferred catalyst and support, loading, and temperatures as discussed above are summarized in Table 9 below. The numerical ranges set forth in Table 9 below are understood to be prefaced by “about”.

TABLE 9
Summary of Palladium on Alumina Support

| Catalyst | Support | Loading (%) | Temperature (° C.) |
|---|---|---|---|
| Pd | alpha(α)-Al₂O₃ | 0.1-0.5 | 75-250 |
| Pd | alpha(α)-Al₂O₃ | 0.1-0.4 | 75-250 |
| Pd | alpha(α)-Al₂O₃ | 0.1-0.3 | 75-250 |
| Pd | alpha(α)-Al₂O₃ | 0.2-0.5 | 75-250 |
| Pd | alpha(α)-Al₂O₃ | 0.2-0.4 | 75-250 |
| Pd | alpha(α)-Al₂O₃ | 0.2-0.3 | 75-250 |
| Pd | alpha(α)-Al₂O₃ | 0.1-0.5 | 100-200 |
| Pd | alpha(α)-Al₂O₃ | 0.1-0.4 | 100-200 |
| Pd | alpha(α)-Al₂O₃ | 0.1-0.3 | 100-200 |
| Pd | alpha(α)-Al₂O₃ | 0.2-0.5 | 100-200 |
| Pd | alpha(α)-Al₂O₃ | 0.2-0.4 | 100-200 |
| Pd | alpha(α)-Al₂O₃ | 0.2-0.3 | 100-200 |
| Pd | alpha(α)-Al₂O₃ | 0.1-0.5 | 100-150 |
| Pd | alpha(α)-Al₂O₃ | 0.1-0.4 | 100-150 |
| Pd | alpha(α)-Al₂O₃ | 0.1-0.3 | 100-150 |
| Pd | alpha(α)-Al₂O₃ | 0.2-0.5 | 100-150 |
| Pd | alpha(α)-Al₂O₃ | 0.2-0.4 | 100-150 |
| Pd | alpha(α)-Al₂O₃ | 0.2-0.3 | 100-150 |
| Pd | theta(θ)-Al₂O₃ | 0.1-0.5 | 100-225 |
| Pd | theta(θ)-Al₂O₃ | 0.1-0.4 | 100-225 |
| Pd | theta(θ)-Al₂O₃ | 0.1-0.3 | 100-225 |
| Pd | theta(θ)-Al₂O₃ | 0.2-0.5 | 100-225 |
| Pd | theta(θ)-Al₂O₃ | 0.2-0.4 | 100-225 |
| Pd | theta(θ)-Al₂O₃ | 0.2-0.3 | 100-225 |
| Pd | theta(θ)-Al₂O₃ | 0.1-0.5 | 125-175 |
| Pd | theta(θ)-Al₂O₃ | 0.1-0.4 | 125-175 |
| Pd | theta(θ)-Al₂O₃ | 0.1-0.3 | 125-175 |
| Pd | theta(θ)-Al₂O₃ | 0.2-0.5 | 125-175 |
| Pd | theta(θ)-Al₂O₃ | 0.2-0.4 | 125-175 |
| Pd | theta(θ)-Al₂O₃ | 0.2-0.3 | 125-175 |
| Pd | theta(θ)-Al₂O₃ | 0.1-0.5 | 130-150 |
| Pd | theta(θ)-Al₂O₃ | 0.1-0.4 | 130-150 |
| Pd | theta(θ)-Al₂O₃ | 0.1-0.3 | 130-150 |
| Pd | theta(θ)-Al₂O₃ | 0.2-0.5 | 130-150 |
| Pd | theta(θ)-Al₂O₃ | 0.2-0.4 | 130-150 |
| Pd | theta(θ)-Al₂O₃ | 0.2-0.3 | 130-150 |
| Pd | delta(δ)-Al₂O₃ | 0.1-0.5 | 100-225 |
| Pd | delta(δ)-Al₂O₃ | 0.1-0.4 | 100-225 |
| Pd | delta(δ)-Al₂O₃ | 0.1-0.3 | 100-225 |
| Pd | delta(δ)-Al₂O₃ | 0.2-0.5 | 100-225 |
| Pd | delta(δ)-Al₂O₃ | 0.2-0.4 | 100-225 |
| Pd | delta(δ)-Al₂O₃ | 0.2-0.3 | 100-225 |
| Pd | delta(δ)-Al₂O₃ | 0.1-0.5 | 125-175 |
| Pd | delta(δ)-Al₂O₃ | 0.1-0.4 | 125-175 |
| Pd | delta(δ)-Al₂O₃ | 0.1-0.3 | 125-175 |
| Pd | delta(δ)-Al₂O₃ | 0.2-0.5 | 125-175 |
| Pd | delta(δ)-Al₂O₃ | 0.2-0.4 | 125-175 |
| Pd | delta(δ)-Al₂O₃ | 0.2-0.3 | 125-175 |
| Pd | delta(δ)-Al₂O₃ | 0.1-0.5 | 130-155 |
| Pd | delta(δ)-Al₂O₃ | 0.1-0.4 | 130-155 |
| Pd | delta(δ)-Al₂O₃ | 0.1-0.3 | 130-155 |
| Pd | delta(δ)-Al₂O₃ | 0.2-0.5 | 130-155 |
| Pd | delta(δ)-Al₂O₃ | 0.2-0.4 | 130-155 |
| Pd | delta(δ)-Al₂O₃ | 0.2-0.3 | 130-155 |
| Pd | alpha(α)-Al₂O₃ | 0.01-10 | 75-250 |
| Pd | theta(θ)-Al₂O₃ | 0.01-10 | 75-250 |
| Pd | delta(δ)-Al₂O₃ | 0.01-10 | 75-250 |
| Pd | activated carbon | 0.01-10 | 75-250 |
| Pd | alpha(α)-Al₂O₃ | 0.05-2 | 75-250 |
| Pd | theta(θ)-Al₂O₃ | 0.05-2 | 75-250 |
| Pd | delta(δ)-Al₂O₃ | 0.05-2 | 75-250 |
| Pd | activated carbon | 0.05-2 | 75-250 |
| Pd | alpha(α)-Al₂O₃ | 0.1-1 | 75-250 |
| Pd | theta(θ)-Al₂O₃ | 0.1-1 | 75-250 |
| Pd | delta(δ)-Al₂O₃ | 0.1-1 | 75-250 |
| Pd | activated carbon | 0.1-1 | 75-250 |
| Pt | alpha(α)-Al₂O₃ | 0.01-10 | 75-250 |
| Pt | theta(θ)-Al₂O₃ | 0.01-10 | 75-250 |
| Pt | delta(δ)-Al₂O₃ | 0.01-10 | 75-250 |
| Pt | activated carbon | 0.01-10 | 75-250 |
| Pt | alpha(α)-Al₂O₃ | 0.05-2 | 75-250 |
| Pt | theta(θ)-Al₂O₃ | 0.05-2 | 75-250 |
| Pt | delta(δ)-Al₂O₃ | 0.05-2 | 75-250 |
| Pt | activated carbon | 0.05-2 | 75-250 |
| Pt | alpha(α)-Al₂O₃ | 0.1-1 | 75-250 |
| Pt | theta(θ)-Al₂O₃ | 0.1-1 | 75-250 |
| Pt | delta(δ)-Al₂O₃ | 0.1-1 | 75-250 |
| Pt | activated carbon | 0.1-1 | 75-250 |
| Pt | alpha(α)-Al₂O₃ | 0.2-0.5 | 75-250 |
| Pt | theta(θ)-Al₂O₃ | 0.2-0.5 | 75-250 |
| Pt | delta(δ)-Al₂O₃ | 0.2-0.5 | 75-250 |
| Pt | activated carbon | 0.2-0.5 | 75-250 |
| Rh | alpha(α)-Al₂O₃ | 0.01-10 | 75-250 |
| Rh | theta(θ)-Al₂O₃ | 0.01-10 | 75-250 |
| Rh | delta(δ)-Al₂O₃ | 0.01-10 | 75-250 |
| Rh | activated carbon | 0.01-10 | 75-250 |
| Rh | alpha(α)-Al₂O₃ | 0.05-2 | 75-250 |
| Rh | theta(θ)-Al₂O₃ | 0.05-2 | 75-250 |
| Rh | delta(δ)-Al₂O₃ | 0.05-2 | 75-250 |
| Rh | activated carbon | 0.05-2 | 75-250 |
| Rh | alpha(α)-Al₂O₃ | 0.1-1 | 75-250 |
| Rh | theta(θ)-Al₂O₃ | 0.1-1 | 75-250 |
| Rh | delta(δ)-Al₂O₃ | 0.1-1 | 75-250 |
| Rh | activated carbon | 0.1-1 | 75-250 |
| Rh | alpha(α)-Al₂O₃ | 0.2-0.5 | 75-250 |
| Rh | theta(θ)-Al₂O₃ | 0.2-0.5 | 75-250 |
| Rh | delta(δ)-Al₂O₃ | 0.2-0.5 | 75-250 |
| Rh | activated carbon | 0.2-0.5 | 75-250 |
| Ru | alpha(α)-Al₂O₃ | 0.01-10 | 75-250 |
| Ru | theta(θ)-Al₂O₃ | 0.01-10 | 75-250 |
| Ru | delta(δ)-Al₂O₃ | 0.01-10 | 75-250 |
| Ru | activated carbon | 0.01-10 | 75-250 |
| Ru | alpha(α)-Al₂O₃ | 0.05-2 | 75-250 |
| Ru | theta(θ)-Al₂O₃ | 0.05-2 | 75-250 |
| Ru | delta(δ)-Al₂O₃ | 0.05-2 | 75-250 |
| Ru | activated carbon | 0.05-2 | 75-250 |
| Ru | alpha(α)-Al₂O₃ | 0.1-1 | 75-250 |
| Ru | theta(θ)-Al₂O₃ | 0.1-1 | 75-250 |
| Ru | delta(δ)-Al₂O₃ | 0.1-1 | 75-250 |
| Ru | activated carbon | 0.1-1 | 75-250 |
| Ru | alpha(α)-Al₂O₃ | 0.2-0.5 | 75-250 |
| Ru | theta(θ)-Al₂O₃ | 0.2-0.5 | 75-250 |
| Ru | delta(δ)-Al₂O₃ | 0.2-0.5 | 75-250 |
| Ru | activated carbon | 0.2-0.5 | 75-250 |
| Ir | alpha(α)-Al₂O₃ | 0.01-10 | 75-250 |
| Ir | theta(θ)-Al₂O₃ | 0.01-10 | 75-250 |
| Ir | delta(δ)-Al₂O₃ | 0.01-10 | 75-250 |
| Ir | activated carbon | 0.01-10 | 75-250 |
| Ir | alpha(α)-Al₂O₃ | 0.05-2 | 75-250 |
| Ir | theta(θ)-Al₂O₃ | 0.05-2 | 75-250 |
| Ir | delta(δ)-Al₂O₃ | 0.05-2 | 75-250 |
| Ir | activated carbon | 0.05-2 | 75-250 |
| Ir | alpha(α)-Al₂O₃ | 0.1-1 | 75-250 |
| Ir | theta(θ)-Al₂O₃ | 0.1-1 | 75-250 |
| Ir | delta(δ)-Al₂O₃ | 0.1-1 | 75-250 |
| Ir | activated carbon | 0.1-1 | 75-250 |
| Ir | alpha(α)-Al₂O₃ | 0.2-0.5 | 75-250 |
| Ir | theta(θ)-Al₂O₃ | 0.2-0.5 | 75-250 |
| Ir | delta(δ)-Al₂O₃ | 0.2-0.5 | 75-250 |
| Ir | activated carbon | 0.2-0.5 | 75-250 |
| Fe | alpha(α)-Al₂O₃ | 5-80 | 75-250 |
| Fe | theta(θ)-Al₂O₃ | 5-80 | 75-250 |
| Fe | delta(δ)-Al₂O₃ | 5-80 | 75-250 |
| Fe | activated carbon | 5-80 | 75-250 |
| Fe | alpha(α)-Al₂O₃ | 20-30 | 75-250 |
| Fe | theta(θ)-Al₂O₃ | 20-30 | 75-250 |
| Fe | delta(δ)-Al₂O₃ | 20-30 | 75-250 |
| Fe | activated carbon | 20-30 | 75-250 |
| Co | alpha(α)-Al₂O₃ | 5-80 | 75-250 |
| Co | theta(θ)-Al₂O₃ | 5-80 | 75-250 |
| Co | delta(δ)-Al₂O₃ | 5-80 | 75-250 |
| Co | activated carbon | 5-80 | 75-250 |
| Co | alpha(α)-Al₂O₃ | 20-30 | 75-250 |
| Co | theta(θ)-Al₂O₃ | 20-30 | 75-250 |
| Co | delta(δ)-Al₂O₃ | 20-30 | 75-250 |
| Co | activated carbon | 20-30 | 75-250 |
| Ni | alpha(α)-Al₂O₃ | 5-80 | 75-250 |
| Ni | theta(θ)-Al₂O₃ | 5-80 | 75-250 |
| Ni | delta(δ)-Al₂O₃ | 5-80 | 75-250 |
| Ni | activated carbon | 5-80 | 75-250 |
| Ni | alpha(α)-Al₂O₃ | 20-30 | 75-250 |
| Ni | theta(θ)-Al₂O₃ | 20-30 | 75-250 |
| Ni | delta(δ)-Al₂O₃ | 20-30 | 75-250 |
| Ni | activated carbon | 20-30 | 75-250 |

### Products—Selectivity

For reactions using each catalyst/support combination (each row) in Table 1, the hydrogenation step (Step (i)) may produce a product mixture in the reactor comprising of the desired product 1,1,2-trifluoroethane (HFC-143); desired intermediates, such as 1-chloro-1,1,2-trifluoroethane (HCFC-133b), 1-chloro-1,2,2-trifluoroethane (HCFC-133), and trifluoroethylene (HFO-1123); and undesired byproducts, such as ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,1-trifluoroethane (HFC-143a), etc.

It has been discovered that the intermediates including 1-chloro-1,1,2-trifluoroethane (HCFC-133b), 1-chloro-1,2,2-trifluoroethane (HCFC-133), and/or trifluoroethylene (HFO-1123) are formed in Step (i), and further, that each of 1-chloro-1,1,2-trifluoroethane (HCFC-133b), 1-chloro-1,2,2-trifluoroethane (HCFC-133), and/or trifluoroethylene (HFO-1123) may themselves be converted to 1,1,2-trifluoroethane (HFC-143). In view of this finding, 1-chloro-1,1,2-trifluoroethane (HCFC-133b), 1-chloro-1,2,2-trifluoroethane (HCFC-133), and/or trifluoroethylene (HFO-1123) are considered “desirable” intermediates because each of the foregoing may be converted to the desired product 1,1,2-trifluoroethane (HFC-143) in order to increase the overall efficiency of the process.

Additionally, it has further been discovered that undesired byproducts such as ethane (HC-170), chloroethane (HCC-160), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), 1,1,1-trifluoroethane (HFC-143a), etc. may also be formed in Step (i) and yet are not able to be converted to form 1,1,2-trifluoroethane (HFC-143). Therefore, the present reaction methods and conditions are tailored to avoid and/or minimize formation of such undesired byproducts.

As demonstrated by the Examples herein, for reactions using each catalyst/support combination (each row) in Table 1, the hydrogenation step may achieve a selectivity to the desired product 1,1,2-trifluoroethane (HFC-143) of from about 20% to about 93%, or from about 30% to about 93%, or from about 40% to about 93%, or from about 50% to about 93%, or from about 60% to about 93%, or from about 70% to about 93%, or from about 80% to about 93%, or from about 90% to about 93%, alternatively the selectivity to the 1,1,2-trifluoroethane (HFC-143) product is greater than about 20%, greater than about 30%, greater than about 40%, greater than about 50%, greater than about 60%, greater than about 70%, greater than about 80%, greater than about 90%, and for each of the foregoing, less than or equal to about 93%, or within any range encompassed by two of the foregoing values as endpoints, based on total moles of the organic components of the composition. Specific examples of additional suitable ranges are set forth below in Table 10. The numerical ranges set forth in Table 10 below are understood to be prefaced by “about”.

TABLE 10
Selectivity to Desired Product using Catalysts
(Pd, Pt, Rh, Ru, Ir, Fe, Co, or Ni) on Supports
(alpha(α)-Al₂O₃, theta(θ)-Al₂O₃, or delta(δ)-
Al₂O₃ or activated carbon) - Each Catalyst/Support
Combination (Each Row) in Table 1 - Step (i) Hydrogenation Reaction

| From (%) | To (%) |
|---|---|
| 20 | 93 |
| 30 | 93 |
| 40 | 93 |
| 50 | 93 |
| 60 | 93 |
| 70 | 93 |
| 80 | 93 |
| 90 | 93 |
| 35 | 40 |
| 40 | 50 |
| 50 | 60 |
| 60 | 70 |
| 70 | 80 |
| 80 | 90 |

When a palladium catalyst is used on an alpha alumina support, the hydrogenation step may achieve a selectivity to the 1,1,2-trifluoroethane (HFC-143) product of greater than about 20%, greater than about 30%, greater than about 40%, greater than about 50%, greater than about 60%, greater than about 70%, greater than about 80%, greater than about 84%, greater than about 87%, preferably greater than about 88%, most preferably greater than about 90%, and for each of the foregoing, less than or equal to about 91%, or within any range encompassed by two of the foregoing values as endpoints, based on total moles of the organic components of the composition.

The hydrogenation reaction may also produce several desired intermediates such as 1-chloro-1,2,2-trifluoroethane (HCFC-133), 1-chloro-1,1,2-trifluoroethane (HCFC-133b), and trifluoroethylene (HFO-1123). These intermediates are recyclable and can be eventually converted to 1,1,2-trifluoroethane (HFC-143). As demonstrated by the Examples herein, for reactions using each catalyst/support combination (each row) in Table 1, the hydrogenation step may achieve a combined selectivity and/or selectivity to each of the desired product (e.g., 1,1,2-trifluoroethane (HFC-143)), and desired intermediates (e.g., 1-chloro-1,2,2-trifluoroethane (HCFC-133), 1-chloro-1,1,2-trifluoroethane (HCFC-133b), and trifluoroethylene (HFO-1123)) of from about 30% to about 99%, or from about 40% to about 99%, or from about 50% to about 99%, or from about 60% to about 99%, or from about 70% to about 99%, or from about 80% to about 99%, or from about 90% to about 99%, or from about 95% to about 99%, or from about 96% to about 99%, or from about 97% to about 99%, or from about 98% to about 99%, or alternatively, the selectivity to the desired product and intermediates may be greater than about 30%, greater than about 40%, greater than about 50%, greater than about 60%, greater than about 70%, greater than about 80%, greater than about 90%, greater than about 95%, greater than about 96%, greater than about 97%, greater than about 98%, and for each of the foregoing, less than or equal to about 99%, or within any range encompassed by two of the foregoing values as endpoints, based on total moles of the organic components of the composition. Specific examples of additional suitable ranges are set forth below in Table 11. The numerical ranges set forth in Table 11 below are understood to be prefaced by “about”.

TABLE 11
Combined Selectivity to Desired Product and Intermediates
using Catalysts (Pd. Pt, Rh, Ru, Ir, Fe, Co, or Ni) on on Supports (alpha(α
)-Al₂O₃, theta(θ)-Al₂O₃, or delta(δ)-Al₂O₃ or activated
carbon) - Each Catalyst/Support Combination (Each Row) in
Table 1 - Step (i) Hydrogenation Reaction

| From (%) | To (%) |
|---|---|
| 30 | ≤99 |
| 40 | ≤99 |
| 50 | ≤99 |
| 60 | ≤99 |
| 70 | ≤99 |
| 80 | ≤99 |
| 90 | ≤99 |
| 95 | ≤99 |
| 96 | ≤99 |
| 97 | ≤99 |
| 98 | ≤99 |
| 30 | 40 |
| 40 | 50 |
| 50 | 60 |
| 60 | 70 |
| 70 | 80 |
| 80 | 90 |
| 90 | 95 |
| 95 | 96 |
| 96 | 97 |
| 97 | 98 |

When a palladium catalyst is used on an alpha alumina support, the hydrogenation step may achieve a combined selectivity and/or selectivity to each of 1,1,2-trifluoroethane (HFC-143), 1-chloro-1,2,2-trifluoroethane (HCFC-133), 1-chloro-1,1,2-trifluoroethane (HCFC-133b), and trifluoroethylene (HFO-1123) of greater than about 30%, greater than about 40%, greater than about 50%, greater than about 60%, greater than about 70%, greater than about 80%, greater than about 85%, preferably greater than about 93%, most preferably greater than about 97%, and for each of the foregoing, less than or equal to about 99%, or within any range encompassed by two of the foregoing values as endpoints, based on total moles of the organic components of the composition.

The hydrogenation reaction may also produce several byproducts such as ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and chloroethane (HCC-160), which are the result of dehydrohalogenation side reactions. These byproducts are undesired as they are difficult to recycle or convert to 1,1,2-trifluoroethane (HFC-143).

As demonstrated by the Examples herein, for reactions using each catalyst/support combination (each row) in Table 1, the hydrogenation step may achieve a combined selectivity and/or selectivity to each of the undesired byproducts (e.g., ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and chloroethane (HCC-160)) of from 0% to about 70%, or from 0% to about 50%, or from 0% to about 30%, or from 0% to about 25%, or from 0% to about 20%, or from 0% to about 15%, or from 0% to about 10%, or from 0% to about 9%, or from 0% to about 8%, or from 0% to about 7%, or from 0% to about 6%, or from 0% to about 5%, or from 0% to about 4%, or from 0% to about 3%, or from 0% to about 2%, or alternatively, the selective to the undesired byproducts may be less than about 70%, less than about 50%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, and for each of the foregoing, greater than or equal to 0%, or within any range encompassed by two of the foregoing values as endpoints, based on total moles of the organic components of the composition. Specific examples of additional suitable ranges are set forth below in Table 12. The numerical ranges set forth in Table 12 below are understood to be prefaced by “about”.

TABLE 12
Combined Selectivity to Undesired Byproduct using Catalysts
(Pd, Pt, Rh, Ru, Ir, Fe, Co, or Ni) on Supports (alpha(α)-Al₂O₃, theta(θ
)-Al₂O₃, or delta(δ)-Al₂O₃ or activated carbon) - Each
Catalyst/Support Combination (Each Row) in Table 1 -
Step (i) Hydrogenation Reaction

| From (%) | To (%) |
|---|---|
| ≥0 | 70 |
| ≥0 | 50 |
| ≥0 | 30 |
| ≥0 | 25 |
| ≥0 | 20 |
| ≥0 | 15 |
| ≥0 | 10 |
| ≥0 | 9 |
| ≥0 | 8 |
| ≥0 | 7 |
| ≥0 | 6 |
| ≥0 | 5 |
| ≥0 | 4 |
| ≥0 | 3 |
| ≥0 | 2 |
| ≥0 | 1 |
| 0.05 | 0.1 |
| 0.1 | 0.2 |
| 0.2 | 0.3 |
| 0.3 | 0.4 |
| 0.4 | 0.5 |
| 0.5 | 0.6 |
| 0.6 | 0.7 |
| 0.7 | 0.8 |
| 0.9 | 1 |
| 1 | 2 |
| 2 | 34 |
| 3 | 4 |
| 4 | 5 |
| 5 | 6 |
| 6 | 7 |
| 7 | 8 |
| 8 | 9 |
| 9 | 10 |
| 10 | 15 |
| 15 | 20 |

In connection with Table 11, the hydrogenation step may achieve a selectivity to byproduct ethane (HC-170) of from 0% to about 25%, or from 0% to about 20%, or from 0% to about 15%, or from 0% to about 10%, or from 0% to about 5%, or from 0% to about 4%, or from 0% to about 3%, or from 0% to about 2%, or from 0% to about 1%, or from 0% to about 0.1%, alternatively, the selectivity to byproduct ethane (HC-170) may be less than about 25%, less than about 20%, less than about 15%, less than about 10%, less than about 5%, less than about 4%, less than about 3%, less than about 2%, less than about 1%, less than about 0.1%, and for each of the foregoing, greater than or equal to 0%, or within any range encompassed by two of the foregoing values as endpoints, based on total moles of the organic components of the composition.

In connection with Table 11, the hydrogenation step may achieve a selectivity to byproduct 1,1,1-trifluoroethane (HFC-143a) of from 0% to about 5%, or from 0% to about 4%, or from 0% to about 3%, or from 0% to about 2%, or from 0% to about 1%, or from 0% to about 0.7%, or from 0% to about 0.5%, or from 0% to about 0.4%, alternatively, the selectivity to byproduct 1,1,1-trifluoroethane (HFC-143a) may be less than about 5%, less than about 4%, less than about 3%, less than about 2%, less than about 1%, less than about 0.7%, less than about 0.5%, less than about 0.4%, and for each of the foregoing, greater than or equal to 0%, or within any range encompassed by two of the foregoing values as endpoints, based on total moles of the organic components of the composition.

In connection with Table 11, the hydrogenation step may achieve a selectivity to byproduct 1,1,1,2-tetrafluoroethane (HFC-134a) of from 0% to about 27%, or from 0% to about 20%, or from 0% to about 15%, or from 0% to about 10%, or from 0% to about 5%, or from 0% to about 4%, or from 0% to about 3%, or from 0% to about 2%, or from 0% to about 1%, or from 0% to about 0.5%, or from 0% to about 0.3%, alternatively, the selectivity to byproduct 1,1,1,2-tetrafluoroethane (HFC-134a) may be less than about 27%, less than about 20%, less than about 15%, less than about 10%, less than about 5%, less than about 4%, less than about 3%, less than about 2%, less than about 1%, less than about 0.5%, less than 0.3%, and for each of the foregoing, greater than or equal to 0%, or within any range encompassed by two of the foregoing values as endpoints, based on total moles of the organic components of the composition.

In connection with Table 11, the hydrogenation step may achieve a selectivity to byproduct 1,1-difluoroethane (HFC-152a) of from 0% to about 5%, or from 0% to about 3%, or from 0% to about 2%, or from 0% to about 1.5%, or from 0% to about 1%, or from 0% to about 0.8%, or from 0% to about 0.5%, or from 0% to about 0.4%, or from 0% to about 0.3%, or from 0% to about 0.2%, or from 0% to about 0.1%, or from 0% to about 0.05%, alternatively, the selectivity to byproduct 1,1-difluoroethane (HFC-152a) may be less than about 5%, less than about 3%, less than about 2%, less than about 1.5%, less than about 1%, less than about 0.8%, less than about 0.5%, less than about 0.4%, less than about 0.3%, less than about 0.2%, less than about 0.1%, less than about 0.05%, and for each of the foregoing, greater than or equal to 0%, or within any range encompassed by two of the foregoing values as endpoints, based on total moles of the organic components of the composition.

In connection with Table 11, the hydrogenation step may achieve a selectivity to byproduct HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) of from 0% to about 10%, or from 0% to about 6%, or from 0% to about 5%, or from 0% to about 4%, or from 0% to about 3%, or from 0% to about 2%, or from 0% to about 1%, or from 0% to about 0.6%, or from 0% to about 0.3%, or from 0% to about 0.2%, or from 0% to about 0.1%, alternatively, the selectivity to byproduct HCFC-142 isomers may be less than about 10%, less than about 6%, less than about 5%, less than about 4%, less than about 3%, less than about 2%, less than about 1%, less than about 0.6%, less than about 0.3%, less than about 0.1%, and for each of the foregoing, greater than or equal to 0%, or within any range encompassed by two of the foregoing values as endpoints, based on total moles of the organic components of the composition.

In connection with Table 11, the hydrogenation step may achieve a selectivity to byproduct chloroethane (HCC-160) of from 0% to about 2%, or from 0% to about 1.6%, or from 0% to about 1.4%, or from 0% to about 1%, or from 0% to about 0.9%, or from 0% to about 0.8%, or from 0% to about 0.7%, or from 0% to about 0.6%, or from 0% to about 0.5%, or from 0% to about 0.3%, or from 0% to about 0.1%, alternatively, the selectivity to byproduct chloroethane (HCC-160) may be less than about 2%, less than about 1.6%, less than about 1.4%, less than about 1%, less than about 0.9%, less than about 0.8%, less than about 0.7%, less than about 0.6%, less than about 0.5%, less than about 0.3%, less than about 0.1%, and for each of the foregoing, greater than or equal to 0%, or within any range encompassed by two of the foregoing values as endpoints, based on total moles of the organic components of the composition.

When a palladium catalyst is used on an alpha alumina support, the hydrogenation step may achieve a combined selectivity and/or selectivity to each of ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and chloroethane (HCC-160) of from about 0% to about 30%, or from 0% to about 25%, or from 0% to about 20%, or from 0% to about 16%, or from 0% to about 12%, or from 0% to about 7%, or from 0% to about 6%, or from 0% to about 3%, alternatively, the combined selectivity and/or selectivity to each of the undesired byproduct may be less than about 30%, less than about 25%, less than about 20%, less than about 16%, less than about 12%, less than about 7%, preferably less than about 6%, most preferably less than about 3%, and for each of the foregoing, greater than or equal to 0%, or within any range encompassed by two of the foregoing values as endpoints, based on total moles of the organic components of the composition.

### Products—Conversion

For reactions using each catalyst/support combination (each row) in Table 1, as also demonstrated by the Examples herein, the hydrogenation step may achieve a conversion of chlorotrifluoroethylene (CTFE) and/or trifluoroethylene (HFO-1123) of from about 10% to 100%, or from about 20% to 100%, or from about 30% to 100%, or from about 40% to 100%, or from about 50% to 100%, or from about 60% to 100%, or from about 70% to 100%, or from about 75% to 100%, or from about 80% to 100%, or from about 90% to 100%, or from about 95% to 100%, or from about 97% to 100%, or from about 98% to 100%, or from about 99% to 100%, or from about 99.9% to 100%, alternatively, the hydrogenation step may achieve a conversion of chlorotrifluoroethylene (CTFE) and/or trifluoroethylene (HFO-1123) of greater than about 10%, greater than about 20%, greater than about 30%, greater than about 40%, greater than about 50% greater than about 60%, greater than about 70%, greater than about 75%, greater than about 80%, greater than about 90%, greater than about 95%, greater than about 97%, greater than about 98%, greater than about 99%, greater than about 99.9%, or of 100%, and for each of the foregoing, less than or equal to 100%, or within any range encompassed by two of the foregoing values as endpoints, based on total moles of the organic components of the composition. Specific examples of additional suitable ranges are set forth below in Table 13. The numerical ranges set forth in Table 13 below are understood to be prefaced by “about”.

TABLE 13
Conversion of CTFE and HFO-1123 using Catalysts (Pd,
Pt, Rh, Ru, Ir, Fe, Co, or Ni) on Supports (alpha(α)-Al₂O₃,
or theta(θ)-Al₂O₃, or delta(δ)-Al₂O₃)

| From (%) | To (%) |
|---|---|
| 10 | ≤100 |
| 20 | ≤100 |
| 30 | ≤100 |
| 40 | ≤100 |
| 50 | ≤100 |
| 60 | ≤100 |
| 70 | ≤100 |
| 75 | ≤100 |
| 80 | ≤100 |
| 90 | ≤100 |
| 95 | ≤100 |
| 97 | ≤100 |
| 98 | ≤100 |
| 99 | ≤100 |
| 99.9 | ≤100 |
| 90 | 95 |
| 95 | 98 |
| 98 | 99 |
| 99 | 99.9 |

When a palladium catalyst is used on an alpha alumina support, the hydrogenation step may achieve a conversion of chlorotrifluoroethylene (CTFE) and/or trifluoroethylene (HFO-1123) of from about 10% to 100%, or from about 20% to 100%, or from about 30% to 100%, or from about 40% to 100%, or from about 50% to 100%, or from about 60% to 100%, or from about 70% to 100%, or from about 75% to 100%, or from about 80% to 100%, or from about 90% to 100%, or from about 95% to 100%, or from about 97% to 100%, or from about 98% to 100%, or from about 99% to 100%, or from about 99.9% to 100%, alternatively, the hydrogenation step may achieve a conversion of chlorotrifluoroethylene (CTFE) and/or trifluoroethylene (HFO-1123) of greater than about 10%, greater than about 20%, greater than about 30%, greater than about 40%, greater than about 50%, greater than about 60%, greater than about 70%, greater than about 80%, greater than about 84%, greater than about 85%, greater than about 87%, greater than about 88%, greater than about 90%, greater than about 95%, preferably greater than about 97%, more preferably greater than about 99%, and most preferably of 100%, and for each of the foregoing, less than or equal to 100%, or within any range encompassed by two of the foregoing values as endpoints, based on total moles of the organic components of the composition.

### Regeneration of Catalyst

For reactions using each catalyst/support combination (each row) in Table 1, it may be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days, or within any range encompassed by two of the foregoing values as endpoints. This may be followed by hydrogen treatment at temperatures of from about 100° C. to about 400° C., preferably from about 200° C. to about 300° C. for carbon and alumina supported metal catalysts.

### Palladium on Alumina Support

When a palladium catalyst is used on an alumina support for a hydrogenation reaction of Step (i), one or more of the following properties may be present. The loading of palladium on the support may be from about 0.01 wt. % to about 5 wt. %, preferably from about 0.05 wt. % to about 2 wt. %, and more preferably from about 0.1 wt. % to about 1 wt. %. The BET surface area may be from about 1 m²/g to about 500 m²/g, preferably from about 1 m²/g to about 200 m²/g, more preferably from about 1 m²/g to about 100 m²/g, and most preferably from about 1 m²/g to about 20 m²/g. The catalyst may be dried at a temperature of from about 100° C. to about 700° C., preferably from about 120° C. to about 500° C., most preferably from about 120° C. to about 300° C., or within any range encompassed by two of the foregoing values as endpoints.

When a palladium catalyst is used on an alumina support for a hydrogenation reaction of Step (i), the palladium catalyst may be exposed to an inert gas such as N₂ for from about 1 hour to about 20 hours, preferably from about 1 hour to about 10 hours, most preferably, from about 1 hour to about 3 hours. The reaction temperature may be from about 100° C. to about 400° C., preferably from about 100° C. to about 300° C., most preferably from about 150° C. to about 250° C. The contact time may be from about 1 second to about 60 seconds, preferably from about 5 seconds to about 40 seconds, most preferably from about 10 seconds to about 30 seconds. The pressure may be from about 1 psig to about 300 psig, preferably from about 1 psig to about 200 psig, most preferably from about 10 psig to about 100 psig, or within any range encompassed by two of the foregoing values as endpoints.

When a palladium catalyst is used on an alumina support for a hydrogenation reaction of Step (i), the mole ratio of hydrogen to chlorotrifluoroethylene (CTFE) and/or trifluoroethylene (HFO-1123) may be from about 2:1 to about 20:1, preferably from about 3:1 to about 15:1, most preferably from about 4:1 to about 10:1. The hydrogenation step achieves a selectivity to the 1,1,2-trifluoroethane (HFC-143) product of greater than about 80%, preferably greater than about 85%, most preferably greater than about 90%. The hydrogenation step achieves a combined selectivity and/or selectivity to each of 1,1,2-trifluoroethane (HFC-143), 1-chloro-1,2,2-trifluoroethane (HCFC-133), 1-chloro-1,1,2-trifluoroethane (HCFC-133b), and trifluoroethylene (HFO-1123) of greater than about 85%, preferably greater than about 93%, most preferably greater than about 97%, based on total moles of the organic components of the composition.

When a palladium catalyst is used on an alumina support for a hydrogenation reaction of Step (i), the hydrogenation step achieves a combined selectivity and/or selectivity to each of ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and chloroethane (HCC-160) of less than about 10%, preferably less than about 5%, most preferably less than about 3%. The hydrogenation step achieves a conversion of chlorotrifluoroethylene (CTFE) and/or trifluoroethylene (HFO-1123) of greater than about 85%, greater than about 90%, preferably greater than about 95%, more preferably greater than about 97%, and most preferably greater than about 99%, and for each of the foregoing, less than or equal to 100%, based on total moles of the organic components of the composition.

When a palladium catalyst is used on an alumina support for a hydrogenation reaction of Step (i), the product stream of the hydrogeneration step may be sent to a number of unit operations for post-reaction treatments. The product stream is firstly sent to a hydrogen distillation column to separate out unconverted hydrogen for recycling, then to a hydrochloride distillation column to separate out byproduct HCl which can be further treated to obtain a salable HCl product, then to a low pressure caustic (e.g., NaOH) solution scrubber to remove residual acids (HCl and/or HF), then to one or more distillation columns to recover recyclable compounds such as byproduct HFO-1123 and unconverted CTFE and/or trifluoroethylene (HFO-1123) for recycling (i.e., being converted to HFC-143 in hydrogenation reactor), and then to one or more distillation columns to isolate the HFC-143 target product. The said HFC-143 target product has a purity of at least 95%, preferably at least 97%, more preferably at least 99%, and most preferably at least 99.9%, and for each of the foregoing, less than or equal to 100%, based on total moles of the organic components of the product composition.

When a palladium catalyst is used on an alumina support for a hydrogenation reaction of Step (i), the total amount of 1,1,2-trifluoroethane (HFC-143), 1-chloro-1,1,2-trifluoroethane (HCFC-133b), 1-chloro-1,2,2-trifluoroethane (HCFC-133), and trifluoroethylene (HFO-1123) in the product composition is at least 60 mol. %, at least 70 mol. %, at least 80 mol. %, or at least 90 mol. %, or at least 94 mol. %, or at least 96 mol. %, or at least 98 mol. %, and for each of the foregoing, less than or equal to 100 mol. %, based on total moles of the organic components of the product composition.

When a palladium catalyst is used on an alumina support for a hydrogenation reaction of Step (i), the amount of 1,1,2-trifluoroethane (HFC-143) in the product mixture after Step (i) is at least 30 mol. %, at least 50 mol. %, or at least 70 mol. %, or at least 80 mol. %, or at least 90 mol. %, and for each of the foregoing, less than or equal to 100 mol. %, based on the total moles of organic components of the product composition.

When a palladium catalyst is used on an alumina support for a hydrogenation reaction of Step (i), the amount of 1-chloro-1,1,2-trifluoroethane (HCFC-133b) in the product mixture, if present, is less than 10 mol. %, less than 8 mol. %, or less than 4 mol. %, and for each of the foregoing, greater than or equal to 0.01 mol. %, based on the total moles of organic components of the product mixture, for example from 0.1 mol. % to 10 mol. %, from 0.1 mol. % to 8 mol. %, or from 0.1 mol. % to 4 mol. %.

When a palladium catalyst is used on an alumina support for a hydrogenation reaction of Step (i), the amount of 1-chloro-1,2,2-trifluoroethane (HCFC-133) in the product mixture, if present, is less than 10 mol. %, less than 7 mol. %, or less than 3 mol. %, and for each of the foregoing, greater than or equal to 0.01 mol. %, based on the total moles of organic components of the product mixture, for example from 0.1 mol. % to 10 mol. %, from 0.1 mol. % to 7 mol. %, or from 0.1 mol. % to 3 mol. %.

When a palladium catalyst is used on an alumina support for a hydrogenation reaction of Step (i), the amount of trifluoroethylene (HFO-1123) in the product mixture, if present, is less than 30 mol. %, less than 20 mol. %, or less than 10 mol. %, and for each of the foregoing, greater than or equal to 0.01 mol. %, based on the total moles of organic components in the product mixture, for example from 0.1 mol. % to 30 mol. %, from 0.1 mol. % to 20 mol. %, or from 0.1 mol. % to 10 mol. %.

When a palladium catalyst is used on an alumina support for a hydrogenation reaction of Step (i), for example, in the product composition, the amount of 1,1,2-trifluoroethane (HFC-143) in the product composition may be at least 50 mol. % and less than or equal to 99.97 mol. %, and the combine amount of desired intermediates (e.g., HCFC-133b, HCFC-133, and HFO-1123) may be at least 0.03 mol. % and less than or equal to 50 mol. % including, for example, an amount of 1-chloro-1,1,2-trifluoroethane (HCFC-133b) of at least 0.01 mol. % and less than or equal to 10 mol. %, an amount of 1-chloro-1,2,2-trifluoroethane (HCFC-133) of at least 0.01 mol. % and less than or equal to 10 mol. %, and an amount of trifluoroethylene (HFO-1123) of at least 0.01 mol. % and less than or equal to 30 mol. %, based on total moles of HFC-143, HCFC-133b, HCFC-133, and HFO-1123 of the product composition.

The combined amount of HFC-143, HCFC-133b, HCFC-133, and HFO-1123 in the composition set forth in the preceding paragraph may be at least 70 mol. % and less than or equal to 100% of the total moles of organic components of the product composition, while the combined amount of other components including undesired byproducts (e.g., HC-170, HCC-160, HCFC-142 isomers (e.g., HCFC-142a, HCFC-142, and/or HCFC-142b), and HFC-143a) may be greater than or equal to 0 mol. % and less than 15 mol. % of the total moles of organic components of the product composition.

When a palladium catalyst is used on an alumina support for a hydrogenation reaction of Step (i), for example, in the product composition, the amount of 1,1,2-trifluoroethane (HFC-143) in the product composition may be at least 70 mol. % and less than or equal to 99.97 mol. %, and the combine amount of desired intermediates (e.g., HCFC-133b, HCFC-133, and HFO-1123) may be at least 0.03 mol. % and less than or equal to 30 mol. % including, for example, an amount of 1-chloro-1,1,2-trifluoroethane (HCFC-133b) of at least 0.01 mol. % and less than or equal to 8 mol. %, an amount of 1-chloro-1,2,2-trifluoroethane (HCFC-133) of at least 0.01 mol. % and less than or equal to 7 mol. %, and an amount of trifluoroethylene (HFO-1123) of at least 0.01 mol. % and less than or equal to 15 mol. %, based on total moles of HFC-143, HCFC-133b, HCFC-133, and HFO-1123 of the product composition.

The combined amount of HFC-143, HCFC-133b, HCFC-133, and HFO-1123 in the composition set forth in the preceding paragraph may be at least 80 mol. % and less than or equal to 100% of the total moles of organic components of the product composition, while the combined amount of other components including undesired byproducts (e.g., HC-170, HCC-160, HCFC-142 isomers (e.g., HCFC-142a, HCFC-142, and/or HCFC-142b), and HFC-143a) may be greater than or equal to 0 mol. % and less than 10 mol. % of the total moles of organic components of the product composition.

When a palladium catalyst is used on an alumina support for a hydrogenation reaction of Step (i), for example, in the product composition, the amount of 1,1,2-trifluoroethane (HFC-143) in the product composition may be at least 83 mol. % and less than or equal to 99.97 mol. %, and the combine amount of desired intermediates (e.g., HCFC-133b, HCFC-133, and HFO-1123) may be at least 0.03 mol. % and less than or equal to 17 mol. % including, for example, an amount of 1-chloro-1,1,2-trifluoroethane (HCFC-133b) of at least 0.01 mol. % and less than or equal to 4 mol. %, an amount of 1-chloro-1,2,2-trifluoroethane (HCFC-133) of at least 0.01 mol. % and less than or equal to 3 mol. %, and an amount of trifluoroethylene (HFO-1123) of at least 0.01 mol. % and less than or equal to 10 mol. %, based on total moles of HFC-143, HCFC-133b, HCFC-133, and HFO-1123 of the product composition.

The combined amount of HFC-143, HCFC-133b, HCFC-133, and HFO-1123 in the composition set forth in the preceding paragraph may be at least 90 mol. % and less than or equal to 100% of the total moles of organic components of the product composition, while the combined amount of other components including undesired byproducts (e.g., HC-170, HCC-160, HCFC-142 isomers (e.g., HCFC-142a, HCFC-142, and/or HCFC-142b), and HFC-143a) may be greater than or equal to 0 mol. % and less than 5 mol. % of the total moles of organic components of the product composition.

Further, when a palladium catalyst is used on an alumina support, the reaction (i.e., Step (i) hydrogenation reaction of the process) produces a product composition comprising 1,1,2-trifluoroethane (HFC-143) in which one or more undesired byproducts including ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), and/or chloroethane (HCC-160) is minimized.

When a palladium catalyst is used on an alumina support for a hydrogenation reaction of Step (i), the total amount of byproducts including ethane (HC-170), 1,1,1-trifluoroethane (HFC-143a), 1,1,1,2-tetrafluoroethane (HFC-134a), 1,1-difluoroethane (HFC-152a), HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)), chloroethane (HCC-160), and/or other undesired or unidentified byproducts, if present, is less than 70 mol. %, less than 50 mol. %, less than 30 mol. %, less than 20 mol. %, less than 10 mol. %, less than 6 mol. %, less than 4 mol. %, or less than 2 mol. %, and for each of the foregoing, greater than or equal to 0 mol. %, based on total moles of the organic components of the composition.

When a palladium catalyst is used on an alumina support for a hydrogenation reaction of Step (i), the amount of ethane (HC-170) in the product composition is less than 15 mol. %, less than 5 mol. %, less than 1 mol. %, or less than 0.1 mol. %, and for each of the foregoing, greater than or equal to 0 mol. %, based on total moles of the organic components of the composition.

When a palladium catalyst is used on an alumina support for a hydrogenation reaction of Step (i), the amount of 1,1,1-trifluoroethane (HFC-143a) in the product composition is less than 5 mol. %, less than 2 mol. %, less than 1 mol. %, or less than 0.5 mol. %, and for each of the foregoing, greater than or equal to 0 mol. %, based on total moles of the organic components of the composition.

When a palladium catalyst is used on an alumina support for a hydrogenation reaction of Step (i), the amount of 1,1,1,2-tetrafluoroethane (HFC-134a) in the product composition is less than 15 mol. %, less than 10 mol. %, less than 5 mol. %, or less than 1 mol. %, and for each of the foregoing, greater than or equal to 0 mol. %, based on total moles of the organic components of the composition.

When a palladium catalyst is used on an alumina support for a hydrogenation reaction of Step (i), the amount of 1,1-difluoroethane (HFC-152a) in the product composition is less than 4 mol. %, less than 2 mol. %, less than 1 mol. %, or less than 0.5 mol. %, and for each of the foregoing, greater than or equal to 0 mol. %, based on total moles of the organic components of the composition.

When a palladium catalyst is used on an alumina support for a hydrogenation reaction of Step (i), the amount of HCFC-142 isomers (e.g., 1-chloro-1,2-difluoroethane (HCFC-142a), 1-chloro-2,2-difluoroethane (HCFC-142), and/or 1-chloro-1,1-difluoroethane (HCFC-142b)) in the product composition is less than 6 mol. %, less than 2 mol. %, less than 1 mol. %, or less than 0.5 mol. %, and for each of the foregoing, greater than or equal to 0 mol. %, based on total moles of the organic components of the composition.

When a palladium catalyst is used on an alumina support for a hydrogenation reaction of Step (i), the amount of chloroethane (HCC-160) in the product composition is less than 2 mol. %, less than 1 mol. %, less than 0.5 mol. %, or less than 0.1 mol. %, and for each of the foregoing, greater than or equal to 0 mol. %, based on total moles of the organic components of the composition.

When a palladium catalyst is used on an alumina support for a hydrogenation reaction of Step (i), the amount of other undesired or unidentified byproducts in the product composition is less than 5 mol. %, less than 2 mol. %, less than 1 mol. %, or less than 0.5 mol. %, and for each of the foregoing, greater than or equal to 0 mol. %, based on total moles of the organic components of the composition.

When a palladium catalyst is used on an alumina support for a hydrogenation reaction of Step (i), for example, in the product composition, the amount of ethane (HC-170) is greater than or equal to 0 mol. % and less than about 15 mol. %, the amount of 1,1,1-trifluoroethane (HFC-143a) is greater than or equal to 0 mol. % and less than about 5 mol. %, the amount of 1,1,1,2-tetrafluoroethane (HFC-134a) is greater than or equal to 0 mol. % and less than about 15 mol. %, the amount of 1,1-difluoroethane (HFC-152a) is greater than or equal to 0 mol. % and less than about 4 mol. %, the amount of HCFC-142 isomers is greater than or equal to 0 mol. % and less than about 6 mol. %, the amount of chloroethane (HCC-160) is greater than or equal to 0 mol. % and less than about 2 mol. %, the amount of other undesired byproducts is greater than or equal to 0 mol. % and less than about 5 mol. %, based on total moles of the organic components of the composition.

When a palladium catalyst is used on an alumina support for a hydrogenation reaction of Step (i), for example, in the product composition, in addition to the amount of the desired product HFC-143 and one or more of the desired intermediates (e.g., HCFC-133b, HCFC-133, and HFO-1123) for at least 80 mol. % and less than or equal to 100 mol. % of the total moles of organic components in the product composition, the product composition may further include one or more of the undesired byproducts (e.g., HC-170, HCC-160, HCFC-142 isomers (e.g., HCFC-142a, HCFC-142, and/or HCFC-142b), and HFC-143a), for example, an amount of ethane (HC-170) greater than or equal to 0 mol. % and less than about 5 mol. %, an amount of 1,1,1-trifluoroethane (HFC-143a) greater than or equal to 0 mol. % and less than about 2 mol. %, an amount of 1,1,1,2-tetrafluoroethane (HFC-134a) greater than or equal to 0 mol. % and less than about 10 mol. %, an amount of 1,1-difluoroethane (HFC-152a) greater than or equal to 0 mol. % and less than about 2 mol. %, an amount of HCFC-142 isomers greater than or equal to 0 mol. % and less than about 2 mol. %, an amount of chloroethane (HCC-160) greater than or equal to 0 mol. % and less than about 1 mol. %, and an amount of other undesired byproducts greater than or equal to mol. % and less than about 2 mol. %, based on total moles of the organic components of the composition.

When a palladium catalyst is used on an alumina support for a hydrogenation reaction of Step (i), for example, in the product composition, in addition to the amount of the desired product HFC-143 and one or more of the desired intermediates (e.g., HCFC-133b, HCFC-133, and HFO-1123) for at least 85 mol. % and less than or equal to 100 mol. % of the total moles of organic components in the product composition, the product composition may further include one or more of the undesired byproducts (e.g., HC-170, HCC-160, HCFC-142 isomers (e.g., HCFC-142a, HCFC-142, and/or HCFC-142b), and HFC-143a), for example, an amount of ethane (HC-170) greater than or equal to 0 mol. % and less than about 1 mol. %, an amount of 1,1,1-trifluoroethane (HFC-143a) greater than or equal to 0 mol. % and less than about 1 mol. %, an amount of 1,1,1,2-tetrafluoroethane (HFC-134a) greater than or equal to 0 mol. % and less than about 5 mol. %, an amount of 1,1-difluoroethane (HFC-152a) greater than or equal to 0 mol. % and less than about 1 mol. %, an amount of HCFC-142 isomers greater than or equal to 0 mol. % and less than about 1 mol. %, an amount of chloroethane (HCC-160) greater than or equal to 0 mol. % and less than about 0.5 mol. %, and an amount of other undesired byproducts greater than or equal to 0 mol. % and less than about 1 mol. %, based on total moles of the organic components of the composition.

When a palladium catalyst is used on an alumina support for a hydrogenation reaction of Step (i), for example, in the product composition, in addition to the amount of the desired product HFC-143 and one or more of the desired intermediates (e.g., HCFC-133b, HCFC-133, and HFO-1123) for at least 90 mol. % and less than or equal to 100 mol. % of the total moles of organic components in the product composition, the product composition may further include one or more of the undesired byproducts (e.g., HC-170, HCC-160, HCFC-142 isomers (e.g., HCFC-142a, HCFC-142, and/or HCFC-142b), and HFC-143a), for example, an amount of ethane (HC-170) greater than or equal to 0 mol. % and less than about 0.1 mol. %, an amount of 1,1,1-trifluoroethane (HFC-143a) greater than or equal to 0 mol. % and less than about 0.5 mol. %, an amount of 1,1,1,2-tetrafluoroethane (HFC-134a) greater than or equal to 0 mol. % and less than about 1 mol. %, an amount of 1,1-difluoroethane (HFC-152a) greater than or equal to 0 mol. % and less than about 0.5 mol. %, an amount of HCFC-142 isomers greater than or equal to 0 mol. % and less than about 0.5 mol. %, an amount of chloroethane (HCC-160) greater than or equal to 0 mol. % and less than about 0.1 mol. %, an amount of other undesired byproducts greater than or equal to 0 mol. % and less than about 0.5 mol. %, based on total moles of the organic components of the composition.

The step (i) hydrogenation reaction may be conducted at a temperature from about 75° C. to about 225° C. in the presence of a palladium catalyst supported on an alumina support.

The step (i) hydrogenation reaction may be conducted at a temperature from about 100° C. to about 200° C. in the presence of a palladium catalyst supported on an alumina support.

The step (i) hydrogenation reaction may be conducted at a temperature from about 120° C. to about 180° C. in the presence of a palladium catalyst supported on an alumina support.

The step (i) hydrogenation reaction may be conducted at a temperature from about 75° C. to about 225° C. in the presence of a palladium catalyst supported on an alpha alumina support.

The step (i) hydrogenation reaction may be conducted at a temperature from about 100° C. to about 200° C. in the presence of a palladium catalyst supported on an alpha alumina support.

The step (i) hydrogenation reaction may be conducted at a temperature from about 120° C. to about 180° C. in the presence of a palladium catalyst supported on an alpha alumina support.

The step (i) hydrogenation reaction may be conducted at a temperature from about 75° C. to about 225° C. in the presence of a palladium catalyst supported on a theta alumina support.

The step (i) hydrogenation reaction may be conducted at a temperature from about 100° C. to about 200° C. in the presence of a palladium catalyst supported on a theta alumina support.

The step (i) hydrogenation reaction may be conducted at a temperature from about 120° C. to about 180° C. in the presence of a palladium catalyst supported on a theta alumina support.

The step (i) hydrogenation reaction may be conducted at a temperature from about 75° C. to about 225° C. in the presence of a palladium catalyst supported on a delta alumina support.

The step (i) hydrogenation reaction may be conducted at a temperature from about 100° C. to about 200° C. in the presence of a palladium catalyst supported on a delta alumina support.

The step (i) hydrogenation reaction may be conducted at a temperature from about 120° C. to about 180° C. in the presence of a palladium catalyst supported on a delta alumina support.

### IV. Step (ii)

### General Process—Vapor Phase

The dehydrohalogenation reaction of Step (ii) may be carried out in the vapor phase in a suitable vapor-phase reactor, for example a tubular reactor made from a material which is resistant to temperature and/or corrosion such as nickel and its alloys, including Hastelloy (for example, Hastelloy C276), Inconel (for example Inconel 600), Incoloy, and Monel wherein the vessels which may be lined with fluoropolymers.

The vapor-phase reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Reactants may be flowed through a scrubber to remove byproducts from the rection, such as hydrogen fluoride (HF) and/or hydrogen chloride (HCl), and the reaction products may be collected by capture in a cooled cylinder, for example.

### General Process—Liquid Phase

The dehydrohalogenation reaction of Step (ii) may be carried out in the liquid phase in a suitable liquid-phase reactor, for example a reactor (e.g., a Parr® reactor) made from a material which is resistant to temperature, pressure and/or corrosion such as stainless steel, nickel and its alloys, including Hastelloy (for example, Hastelloy C276), Inconel (for example Inconel 600), Incoloy, and Monel wherein the vessels which may be lined with fluoropolymers.

The liquid-phase reactor may be first cleaned and flushed with deionized water, followed by charging with a basic solution made from metal hydroxides (MOH), for example, alkali metal hydroxides (e.g., LiOH, NaOH, KOH) or alkaline earth metal hydroxides (e.g., Mg(OH)₂, Ca(OH), Sr(OH)₂), or mixtures thereof.

### Catalysts—Vapor Phase

The catalyst and process conditions play an important role in the vapor-phase dehydrohalogenation reaction.

Suitable catalysts for the dehydrohalogenation reaction include metal oxides such as chromium oxide, aluminum oxide, zinc oxide, iron oxide, and magnesium oxide. Fluorination treatment of the metal oxide catalysts may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrohalogenation reaction include metal fluorides such as chromium fluoride, alumina fluoride, zinc fluoride, iron fluoride, magnesium fluoride, and various combinations of thereof.

Other metals, such as Pd, Pt, and Ni, may also be loaded onto the above fluorinated metal oxides, for example, via a wet impregnation process wherein a salt of the metal is exposed to the fluorinated metal oxide support in solution, followed by drying and then reduction with hydrogen gas.

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, based on a total weight of the catalyst and support. For supported noble metal catalysts such as platinum or palladium, the metal loading may be ranged from about 0.01 wt. % to about 5 wt. %, preferably from about 0.05 wt. % to about 2 wt. %, and more preferably from about 0.1 wt. % to about 1 wt. %.

When fluorinated alumina is used, the amount of metal loading on the support may be from about 0.01 wt. % to about 5 wt. %, preferably from about 0.05 wt. % to about 2 wt. %, most preferably from about 0.1 wt. % to about 1 wt. %.

The catalyst used in step (ii) during a vapor phase process may have a proper BET (Brunauer, Emmet, and Teller) surface area. The BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints. For metal oxides catalysts, the BET surface area may be preferably greater than about 100 m²/g. For fluorinated metal oxides catalysts, the BET surface area may be preferably greater than about 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

When fluorinated alumina is used, the BET surface area may be greater than about 10 m²/g, preferably greater than 20 m²/g, most preferably greater than 25 m²/g.

### Catalyst Pretreatment—Vapor Phase

The catalyst for the vapor phase reaction may be pretreated by drying at elevated temperatures, as low as about 200° C., about 250° C., about 300° C., about 350° C., about 360° C., about 370° C., or as high as about 380° C., about 390° C., about 400° C., about 450° C., about 500° C., about 550° C., about 600° C., or within any range encompassed by two of the foregoing values as endpoints. As part of the catalyst activation, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

When fluorinated alumina is used, the vapor-phase catalyst may be pretreated by drying a temperature of from about 200° C. to about 600° C., preferably from about 300° C. to about 600° C., most preferably from about 400° C. to about 550° C.

When fluorinated alumina is used for the vapor phase reaction, the pretreatment process may take from about 1 hour to about 10 hours, preferably from about 2 hours to about 6 hours, most preferably from about 3 hours to about 5 hours.

### Reaction Conditions

The temperature range for dehydrohalogenation reaction may be as low as about 125° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints. The temperature may be preferably from about 250° C. to about 450° C., and more preferably from about 300° C. to about 400° C.

When fluorinated alumina is used, the reaction temperature may be from about 125° C. to about 500° C., preferably from about 250° C. to about 450° C., most preferably from about 300° C. to about 400° C.

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints. For example, the pressure may be from about 1 psig to about 50 psig, preferably from about 5 psig to about 30 psig, and more preferably from about 10 psig to about 20 psig.

When fluorinated alumina is used, the reaction pressure may be from about 1 psig to about 50 psig, preferably from about 5 psig to about 30 psig, most preferably from about 10 psig to about 20 psig.

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be from about 1 second to about 60 seconds.

When fluorinated alumina is used, the contact time may be from about 1 second to about 60 seconds, preferably from about 5 seconds to about 40 seconds, most preferably from about 10 seconds to about 30 seconds.

The reaction may also be conducted substantially in the absence of water. For example, the amount of water present during the reaction may be less than 1 mol. %, less than 0.5 mol. %, or less than 0.05 mol. %, and for each of the foregoing, greater than or equal to 0 mol. %, based on a total weight of the reactants in the reactor.

### Products

In the dehydrohalogenation reactions of Step (ii), the cis/trans molar ratio of the 1,2-difluoroethylene in the product mixture may be as low as about 1:1, about 2:1, about 3:1, about 4:1, about 5:1, about 6:1, or as high as about 7:1, about 8:1, about 9:1, about 10:1, about 11:1, about 12:1, about 13:1, about 14:1, about 15:1 or within any range encompassed by two of the foregoing values as endpoints. For example, the cis/trans ratio may be from about 2:1 to about 15:1.

When fluorinated alumina is used, the cis/trans molar ratio of the 1,2-difluoroethylene in the product mixture may be from about 1:1 to about 15:1, preferably from about 1:1 to about 10:1, most preferably from about 2:1 to about 7:1.

The selectivity to the desired 1,2-difluoroethylene product (the sum of 1232E and 1232Z) may be as low as about 80%, about 85%, about 89%, about 90%, about 91%, about 92%, about 93%, about 94%, about 95% or as high as about 96%, about 97%, about 98%, about 99%, or within any range encompassed by two of the foregoing values as endpoints. For example, the selectivity may be from about 89% to about 99%.

When fluorinated alumina is used, the selectivity to the desired 1,2-difluoroethylene product (the sum of 1232E and 1232Z) may be from about 85% to about 99%, preferably from about 90% to about 99%, most preferably from about 95% to about 99%.

The conversion of the starting material may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, and for each of the foregoing, less than or equal to 100%, or within any range encompassed by two of the foregoing values as endpoints.

When fluorinated alumina is used, the conversion of the starting material may be greater than about 20%, preferably greater than about 30%, most preferably greater than about 60%.

For the vapor phase reaction, it may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C., for fluorinated catalysts or hydrogen treatment at temperatures of from about 100° C. to about 400° C., preferably from about 200° C. to about 350° C. for supported transition metal catalysts.

When fluorinated alumina is used, the catalyst may be regenerated by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C. Additionally, the present process advantageously avoids and/or minimizes formation of 1,1,1,-trifluoroethane (HFC-143a) wherein the products of Step (ii), including trans-1,2-difluoroethylene (HFO-1132E), may include less than 5 wt. %, less than 3 wt. %, less than 1 wt. %, less than 0.5 wt. %, or less than 0.1 wt. %, and for each of the foregoing, greater than or equal to 0 wt. % of 1,1,1,-trifluoroethane (HFC-143a), based on a total weight of the product composition.

When fluorinated alumina is used as a catalyst, the following properties may be present. The amount of metal loading on the support may be from about 0.01 wt. % to about 5 wt. %, preferably from about 0.05 wt. % to about 2 wt. %, most preferably from about 0.1 wt. % to about 1 wt. %. The BET surface area may be greater than about 10 m²/g, preferably greater than 20 m²/g, most preferably greater than 25 m²/g. The catalyst may be pretreated by drying a temperature of from about 200° C. to about 600° C., preferably from about 300° C. to about 600° C., most preferably from about 400° C. to about 550° C. The pretreatment process may take from about 1 hour to about 10 hours, preferably from about 2 hours to about 6 hours, most preferably from about 3 hours to about 5 hours. The reaction temperature may be from about 125° C. to about 500° C., preferably from about 250° C. to about 450° C., most preferably from about 300° C. to about 400° C. The reaction pressure may be from about 1 psig to about 50 psig, preferably from about 5 psig to about 30 psig, most preferably from about 10 psig to about 20 psig. The contact time may be from about 1 second to about 60 seconds, preferably from about 5 seconds to about 40 seconds, most preferably from about 10 seconds to about 30 seconds. The cis/trans molar ration of the 1,2-difluoroethylene in the product mixture may be from about 1 to about 15, preferably from about 1 to about 10, most preferably from about 2 to about 7. The selectivity to the desired 1,2-difluoroethylene product (the sum of 1232E and 1232Z) may be from about 85% to about 99%, preferably from about 90% to about 99%, most preferably from about 95% to about 99%. The conversion of the starting material to 1,2-difluoroethylene may be greater than about 20%, preferably greater than about 30%, most preferably greater than about 60%, and for each of the foregoing, less than or equal to 100%, based on total amount of organic components in the product mixture. The catalyst may be regenerated by passing air or air diluted with nitrogen over the catalyst at temperatures of from about 100° C. to about 400° C., preferably from about 200° C. to about 375° C., for from about 0.5 hour to about 3 days. This regeneration may be followed by hydrogen fluoride treatment at temperatures of from about 100° C. to about 400° C., preferably from about 200° C. to about 350° C.

In the above process, the amount of trans-1,2-difluoroethylene (HFO-1132E) in the product mixture may be at least 5 mol. %, at least 10 mol. %, or at least 20 mol. %, for example, based on the total moles of organic components in the product mixture.

In the above process, the amount of cis-1,2-difluoroethylene (HFO-1132Z) in the product mixture may be at least 60 mol. %, at least 80 mol. %, or at least 90 mol. %, for example, based on the total moles of organic components in the product mixture.

### V. Step (iii)

### General Process

The 1,2-difluoroethylene (HFO-1132) obtained in step (ii) above may be produced as a mixture containing both the trans-1,2-difluoroethylene (HFO-1132E) and cis-1,2-difluoroethylene (HFO-1132Z) isomers.

In step (iii), the cis-1,2-difluoroethylene (HFO-1132Z) isomer may be converted to the trans-1,2-difluoroethylene (HFO-1132E) isomer either by exposure to heat and/or a catalyst to yield a final product comprising, consisting essentially of, or consisting of, the trans-1,2-difluoroethylene (HFO-1132E) isomer in high purity, or substantially high purity.

The isomerization reaction may be conducted in any suitable reaction vessel or reactor, but it should preferably be constructed from materials which are resistant to corrosion such as nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst.

The isomerization reaction may be conducted in a separate reactor from the vapor phase reactor of dehydrohalogenation reactions. Alternatively, the isomerization reaction is carried out in the same vapor phase reactor of dehydrohalogenation reactions. After each reaction steps, distillation columns may be used to separate unconverted raw materials for recycle and target intermediates, byproducts, or products. For example, among components included in the effluent of hydrogenation reactor, CTFE and HFO-1123 may be isolated and sent back to the initial hydrogenation reactor for recycling to improve the process efficiency, whereas HCFC-133 and HFC-143 may be isolated and sent to the dehydrohalogenation reactor for further processing.

Among components included in the effluent of dehydrohalogenation reactor, HCFC-133 and HFC-143 may be isolated and sent back to the dehydrohalogenation reactor for recycling to improve the process efficiency, HFO-1123 may be isolated and sent to the initial hydrogenation reactor for hydrogenation reaction, HFO-1132 (Z) may be isolated and sent to isomerization reactor or dehalogenation reactor for isomerization reaction, and/or HFO-1132 (E) may be isolated and sent to downstream distillation columns for further purification as needed.

### Catalysts

The isomerization reaction may be carried out in a vapor phase reactor charged with isomerization catalysts. Suitable catalysts for the isomerization reaction may include metal oxides such as chromium oxide (Cr₂O₃), aluminum oxide (Al₂O₃), zinc oxide (ZnO), iron oxide (Fe₂O₃), magnesium oxide (MgO), and various combinations of thereof. Fluorination treatment of the metal oxide catalysts may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrohalogenation reaction include metal fluorides such as chromium fluoride (CrF₃), alumina fluoride (AlF₃), zinc fluoride (ZnF₂), iron fluoride (FeF₃), magnesium fluoride (MgF₂), and various combinations of thereof. The main reaction of HFO-1132 (Z)-->HFO-1132 (E) is expected to occur.

### Reaction Conditions

The temperature range for the isomerization reaction may be as low as about 100° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C., or within any range encompassed by two of the foregoing values as endpoints.

The reaction may be conducted at atmospheric pressure, super-atmospheric pressure or under vacuum. The vacuum pressure can be from about 5 torr to about 760 torr. Contact time of the reactants with the catalyst may range from about 0.5 seconds to about 120 seconds, however, longer or shorter times can be used.

The reaction may also be conducted in an inert atmosphere substantially in the absence of oxygen. For example, the amount of oxygen present during the reaction may be less than 10 mol. %, less than 5 mol. %, or less than 1 mol. % based on a total weight of the reactants in the reactor.

The reaction may also be conducted substantially in the absence of water. For example, the amount of water present during the reaction may be less than 1 mol. %, less than 0.5 mol. %, or less than 0.05 mol. %, and for each of the foregoing, greater than or equal to 0 mol. %, based on a total weight of the reactants in the reactor.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### EXAMPLES

### Example 1: Step (i)—Hydrogenation of Chlorotrifluoroethylene (CTFE) to Produce 1,1,2-Trifluoroethane (HFC-143) Using 0.2% Pd/Alpha (α)-Al₂O₃ Catalyst

Example 1 shows conversion of chlorotrifluoroethylene (CTFE) to HFC-143 using H₂ and 0.2% Pd/alpha(α)-Al₂O₃ catalyst. The surface area of the Pd/alpha(α)-Al₂O₃ catalyst used was 2.6 m²/g.

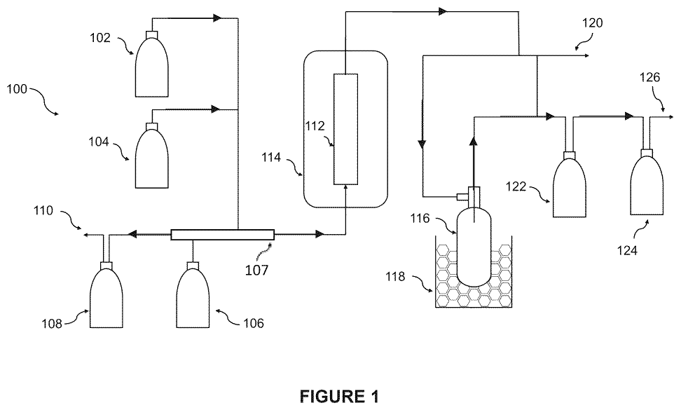

One schematic of a process flow illustrating suitable components for the reaction in step (i) is provided in FIG. 1. Referring to the process flow diagram 100 shown therein, a supply of an inert carrier (e.g., nitrogen (N₂)) is provided from cylinder 102 and a supply of hydrogenation gas (e.g., hydrogen (H₂)) is provided from cylinder 104. The experimental apparatus used for this example includes a feed system containing gas flow controllers for N₂ and H₂ and a Micromotion mass flow meter connected to a research control valve (RCV) controlling the organic flow rate. An organic feedstock (e.g., feed material or reactant composition) which comprises the starting material (e.g., CF₂═CFCl (CTFE) and/or trifluoroethylene (HFO-1123)), is supplied from cylinder 106. Cylinders 102, 104 and 106 are all connected to a scrubber 108 which is coupled to vent 110. A rupture disc 107 is placed between the feed manifold exit (connecting cylinders 102, 104, and 106) and inlet to the reactor 112. The pressure release side of the rupture disc 107 with a burst pressure of 150 psig is connected to the scrubber 108 which is coupled to vent 110. The scrubber 108 is a KOH scrubber. The supply of inert carrier gas, hydrogenation gas, and feed material is fed into reactor 112 which is surrounded by box oven 114.

The reactor consists of a one-inch SS tube packed with the catalyst. A thermocouple is inserted into the middle of the catalyst bed to read the operating temperature. The pressure control system consists of a RCV which is controlling the pressure by getting feedback from the pressure transducer placed after the reactor. The reaction is monitored by taking samples from outlet 120 and conducting a GC analysis. For GC analysis, samples are taken after the reactor using a sample bag filled with 50 ml of water to capture HCl and HF. Before the GC analysis, the sample bag is heated at 60° C. for one hour to assure that all the organic content is in the gas phase. Then, a sample is taken using a syringe and injected into the GC instrument for analysis.

As the reaction proceeds, the products are fed into holding tank 116 which is immersed in a bath of dry ice or a dry ice and acetone mixture 118. The dry ice or dry ice and acetone mixture bath is configured to hold the temperature of holding tank 116 at a reduced temperature below ambient, such as about −87° C., for example. Holding tank 116 is coupled to buffer knock-out tank 122 to prevent potential backflow of the scrubber solution (e.g., KOH solution), which is also coupled to the scrubber 124. Scrubber 124 has a vent 126 which opens to the atmosphere.

Prior to the reactions, the catalyst was pretreated under 150 ml/min of hydrogen (H₂) at 200° C. for four hours in the same reactor. Then, temperature was reduced to 130° C. under flowing hydrogen. When temperature was stable at 130° C., 10 g/h of CTFE was added to the feed stream. Temperature typically increases about 15 to 20° C. upon introduction of CTFE. In Example 1, the temperature is adjusted to about 150° C. if it was different from 150° C.

Table 14 below shows the mole percentages of the product stream after the reactor determined using GC-FID analysis. For the results presented in Table 14, typically three samples at each temperature were collected (one sample every two hours) and then the temperature was changed to the next target. The results are presented in the order reactions were performed.

Since the substrate conversion was 100%, mole percentages are equal to selectivity. As shown in Table 14, initially at 150° C. the selectivity to the R-143 product was 84.61% and the total selectivity to R-143 and recyclable intermediates was 88.21%. The recyclable intermediates observed were R-133 (1.59%), R-133b (1.26%) and R-1123 (0.75%). The major byproducts were R-152a (3.60%), R-170 (1.59%), R-160 (0.88%), R-143a (0.86%), R-142 isomers (0.33%) and R-134a (0.02%).

As shown in Table 14, there was a gradual decrease in product selectivity from 150° C. to 200° C. and then a sharp decline in product selectivity from 200° C. to 252° C. The major byproducts formed at 252° C. were R-134a (39.05%) and R-170 (23.35%). R-170 is a result of complete hydrogenation and R-134a could have formed by addition of HF to the R-1123 intermediate (CHF═CF₂+HF→CH₂F—CF₃). As such, when Step (i) involves use of a Pd/alpha(α)-Al₂O₃ catalyst, higher selectivity towards desired intermediates and R-143 is achieved at a reaction temperature of less than 250° C., more preferably less than about 225° C., or most preferably less than 200° C.

When the temperature was reduced to 151° C., compared to the results obtained on the fresh catalyst at 150° C., a 3.25% increase in R-143 selectivity and 5.62% increase in R-143 plus recyclable intermediates selectivity was observed.

The selectivity to R-143 continuously increased by decreasing the temperature from 151° C. to 100° C. It appears that temperatures as low as 100° C. was enough for complete conversion of CTFE to R-143. Increasing the temperature above 100° C. promoted formation of overhydrogenated products.

In Table 14 below, the organic feed/flow rate was 10 g/h, H₂ feed/flow rate was 150 ml/min, catalyst volume was 50 ml (25 ml of the catalyst was diluted with 25 ml of ⅛″ SS ProPack mesh), and pressure was 45 psig. CTFE conversion was 100% under all conditions in Table 14. The results are presented in the order reactions were performed.

TABLE 14
Mole composition after a reactor packed with 0.2% Pd/alpha(α)-Al₂O₃ and a feed material containing 99.99% pure CTFE

| | | | | | | mol. % (undesired) | | | | | | | |
|---|---|---|---|---|---|---|---|---|---|---|---|---|---|
| Temp | mol. % (desired) | | | | | | | | | R-142 | | | |
| (° C.) | R-143 | R-133b | R-133 | R-1123 | total | R-170 | R-143a | R-134a | R-152a | isomers | R-160 | Other * | total |
| 150 | 84.61 | 1.26 | 1.59 | 0.75 | 88.21 | 1.59 | 0.86 | 0.02 | 3.60 | 0.33 | 0.88 | 4.51 | 11.79 |
| 175 | 82.15 | 1.88 | 2.71 | 0.00 | 86.74 | 4.81 | 2.81 | 0.29 | 1.19 | 1.18 | 1.76 | 1.22 | 13.26 |
| 200 | 79.95 | 2.23 | 2.49 | 0.00 | 84.66 | 4.81 | 4.42 | 0.99 | 0.57 | 1.49 | 1.41 | 1.65 | 15.34 |
| 252 | 22.55 | 2.35 | 3.47 | 0.00 | 28.37 | 23.35 | 4.12 | 39.05 | 0.01 | 1.80 | 0.84 | 2.47 | 71.63 |
| 151 | 87.86 | 3.65 | 2.16 | 0.17 | 93.83 | 1.01 | 1.50 | 0.47 | 2.14 | 0.19 | 0.45 | 0.40 | 6.17 |
| 130 | 88.94 | 3.58 | 2.03 | 0.25 | 94.80 | 0.86 | 1.51 | 0.41 | 1.57 | 0.13 | 0.45 | 0.27 | 5.20 |
| 100 | 90.85 | 2.94 | 1.63 | 2.00 | 97.42 | 0.14 | 0.05 | 0.05 | 1.62 | 0.05 | 0.10 | 0.57 | 2.58 |

* Other includes R-152 (<0.2%), R-133a (<0.1%), R-151 (<0.2%), and unknowns.

### Example 2: Step (i)—Hydrogenation of Chlorotrifluoroethylene (CTFE) to Produce 1,1,2-Trifluoroethane (HFC-143) Using 0.3% Pd/Theta (θ)-Al₂O₃ Catalyst

Example 2 shows conversion of chlorotrifluoroethylene (CTFE) to HFC-143 using H₂ and 0.3% Pd/theta(θ)-Al₂O₃ catalyst. The surface area of the Pd/theta(θ)-Al₂O₃ catalyst used was 38.1 m²/g. The experimental apparatus used for this example were same as Example 1.

Prior to the reactions, the catalyst was pretreated under 150 ml/min of H₂ at 200° C. for four hours in the same reactor. Then, temperature was reduced to 130° C. under flowing hydrogen. When temperature was stable at 130° C., 10 g/h of CTFE was added to the feed stream. Temperature typically increased about 15° C. to 20° C. upon introduction of CTFE. In Example 2, the temperature was adjusted to about 150° C. if it was different from 150° C.

Table 15 below shows the mole percentages of the product stream after the reactor determined using GC-FID analysis. For the results presented in Table 15, typically three samples at each temperature were collected (one sample every two hours) and then the temperature was changed to the next target. The results are presented in the order reactions were performed. Since the substrate conversion was 100%, mole percentages are equal to selectivity.

As shown in Table 15, initially at 150° C. the selectivity to the R-143 product was 83.78% and the total selectivity to R-143 and recyclable intermediates was 90.37%. The recyclable intermediates observed were R-133 (2.75%), R-133b (3.72%) and R-1123 (0.12%). The major byproducts were R-152a (1.19%), R-170 (1.54%), R-160 (0.89%), R-143a (1.63%), R-142 isomers (2.09%) and R-134a (1.03%).

A gradual decrease in product selectivity from 150° C. to 200° C. was observed. The major byproducts formed at 200° C. were R-134a (26.67%) and R-170 (12.33%). R-170 is a result of complete hydrogenation and R-134a could have formed by addition of HF to the R-1123 intermediate (CHF═CF₂+HF→CH₂F—CF₃). As such, when Step (i) involves use of a Pd/theta(θ)-Al₂O₃ catalyst, higher selectivity towards desired intermediates and R-143 is achieved at a reaction temperature of less than 225° C., more preferably less than about 200° C., or most preferably less than 175° C.

When the temperature was reduced to 149° C., compared to the results obtained on the fresh catalyst at 150° C., a 6.61% decrease in R-143 selectivity and 0.03% decrease in R-143 plus recyclable intermediates selectivity was observed.

The selectivity to R-143 increased by decreasing the temperature from 151° C. to 131° C. It appears that temperatures as low as 130° C. was enough for complete conversion of CTFE to R-143. Increasing the temperature above 130° C. promoted formation of overhydrogenated products.

In Table 15 below, the organic feed/flow rate was 10 g/h, H₂ feed/flow rate was 150 ml/min, catalyst volume was 50 ml (25 ml of the catalyst was diluted with 25 ml of ⅛″ SS ProPack mesh), and pressure was 45 psig. CTFE conversion was 100% under all conditions. The results are presented in the order reactions were performed.

TABLE 15
Mole composition after a reactor packed with 0.3% Pd/theta(θ)-
Al₂O₃ and a feed material containing >99.99% pure CTFE

| | | | | | | mol. % (undesired) | | | | | | | |
|---|---|---|---|---|---|---|---|---|---|---|---|---|---|
| Temp | mol. % (desired) | | | | | | | | | R-142 | | | |
| (° C.) | R-143 | R-133b | R-133 | R-1123 | total | R-170 | R-143a | R-134a | R-152a | isomers | R-160 | Other * | total |
| 150 | 83.78 | 3.72 | 2.75 | 0.12 | 90.37 | 1.54 | 1.63 | 1.03 | 1.19 | 2.09 | 0.89 | 1.26 | 9.63 |
| 174 | 75.15 | 5.08 | 4.58 | 0.00 | 84.81 | 3.43 | 2.92 | 5.49 | 0.27 | 1.43 | 0.91 | 0.74 | 15.19 |
| 200 | 44.08 | 4.60 | 4.64 | 0.00 | 53.33 | 12.33 | 2.67 | 26.67 | 0.02 | 2.19 | 1.32 | 1.47 | 46.67 |
| 149 | 77.17 | 7.33 | 5.85 | 0.00 | 90.34 | 1.56 | 1.32 | 4.24 | 0.17 | 1.47 | 0.54 | 0.36 | 9.66 |
| 131 | 82.45 | 7.84 | 6.38 | 0.00 | 96.67 | 0.43 | 0.70 | 0.93 | 0.27 | 0.53 | 0.26 | 0.21 | 3.33 |

* Other includes R-152 (<0.2%), R-133a (<0.1%), R-151 (<0.2%), and unknowns.

### Example 3: Step (i)—Hydrogenation of Chlorotrifluoroethylene (CTFE) to Produce 1,1,2-Trifluoroethane (HFC-143) Using 0.3% Pd/Delta (δ)-Al₂O₃ Catalyst

Example 3 shows conversion of chlorotrifluoroethylene (CTFE) to HFC-143 using H₂ and 0.3% Pd/delta(δ)-Al₂O₃ catalyst. The surface area of this catalyst was 122.4 m²/g. The experimental apparatus used for this example were same as Example 1.

Prior to the reactions, the catalyst was pretreated under 150 ml/min of H₂ at 200° C. for four hours in the same reactor. Then, temperature was reduced to 130° C. under flowing hydrogen. When temperature was stable at 130° C., 10 g/h of CTFE was added to the feed stream. Temperature typically increased about 15° C. to 20° C. upon introduction of the organic. For Example 3, temperature was adjusted to about 150° C. if it was different from 150° C.

Table 16 shows the mole percentages of the product stream after the reactor determined using GC-FID analysis. For the results presented in Table 16, typically three samples at each temperature were collected (one sample every two hours) and then the temperature was changed to the next target. The results are presented in the order reactions were performed. Since the substrate conversion was 100%, mole percentages are equal to selectivity.

As shown in Table 16, initially at 150° C. the selectivity to the R-143 product was 72.48% and the total selectivity to R-143 plus recyclable intermediates was 89.73%. The recyclable intermediates observed were R-133 (2.47%), R-133b (4.86%) and R-1123 (9.92%). The major byproducts were R-152a (0.76%), R-170 (0.63%), R-160 (0.73%), R-143a (0.38%), R-142 isomers (5.93%) and R-134a (0.23%).

A gradual decrease in product selectivity from 150° C. to 200° C. was observed. The major byproducts formed at 200° C. were R-134a (14.68%) and R-170 (4.32%). R-170 was a result of complete hydrogenation and R-134a could have formed by addition of HF to the R-1123 intermediate (CHF═CF₂+HF→CH₂F—CF₃).

When the temperature was reduced to 152° C., compared to the results obtained on the fresh catalyst at 150° C., a 6.53% decrease in R-143 selectivity but 6.20% increase in R-143 plus recyclable intermediates selectivity was observed. The selectivity to R-143 decreased by decreasing the temperature from 152° C. to 130° C. As such, when Step (i) involves use of a Pd/delta(δ)-Al₂O₃ catalyst, higher selectivity towards desired intermediates and R-143 was achieved at a reaction temperature of less than 225° C., more preferably less than about 200° C., or most preferably between about 120° C. and about 180° C.

In Table 16 below, the organic feed/flow rate was 10 g/h, H₂ feed/flow rate was 150 ml/min, catalyst volume was 50 ml (25 ml of the catalyst was diluted with 25 ml of ⅛″ SS ProPack mesh), and pressure was 45 psig. CTFE conversion was 100% under all conditions. The results are presented in the order reactions were performed.

TABLE 16
Mole composition after a reactor packed with 0.3% Pd/delta(δ)-Al₂O₃ and a feed material containing >99.99% pure CTFE

| | | | | | | mol. % (undesired) | | | | | | | |
|---|---|---|---|---|---|---|---|---|---|---|---|---|---|
| Temp | mol. % (desired) | | | | | | | | | R-142 | | | |
| (° C.) | R-143 | R-133b | R-133 | R-1123 | total | R-170 | R-143a | R-134a | R-152a | isomers | R-160 | Other * | total |
| 150 | 72.48 | 4.86 | 2.47 | 9.92 | 89.73 | 0.63 | 0.38 | 0.23 | 0.76 | 5.93 | 0.73 | 1.61 | 10.27 |
| 174 | 60.76 | 5.06 | 2.57 | 18.66 | 87.06 | 1.11 | 1.42 | 3.00 | 0.35 | 5.08 | 0.80 | 1.18 | 12.94 |
| 201** | 37.49 | 4.55 | 2.59 | 27.11 | 71.74 | 4.32 | 2.01 | 14.68 | 0.1 | 0.22 | 1.56 | 5.37 | 28.26 |
| 152 | 65.95 | 4.60 | 1.77 | 23.60 | 95.93 | 0.26 | 0.96 | 1.42 | 0.41 | 0.55 | 0.25 | 0.22 | 4.07 |
| 130 | 62.94 | 3.68 | 1.40 | 30.21 | 98.24 | 0.08 | 0.48 | 0.44 | 0.48 | 0.09 | 0.09 | 0.10 | 1.76 |

* Other includes R-152 (<0.2%), R-133a (<0.1%), R-151 (<0.2%), and unknowns.
**Only 2 hours (1 GC sample) at this temperature. 6 hours (3 GC samples) for the rest of the entries.

### Example 4: Comparing the Three Catalysts in Examples 1-3 at Different Temperatures

Example 4 compares the three catalysts used in Examples 1-3 at different temperatures. The details for the runs are presented in Examples 1-3.

Table 17 shows the product distribution for the first runs at 150° C. on fresh catalysts, pretreated under H₂ at 200° C. The feed material was >99.99% pure CTFE, organic flow rate was 10 g/h, H₂ flow rate was 150 ml/min, and pressure was 45 psig. CTFE conversion was 100%.

As shown in Table 17, the 0.2% Pd/alpha(α)-Al₂O₃ catalyst showed the highest selectivity to R-143. The 0.3% Pd/delta(δ)-Al₂O₃ catalyst showed 9.92% selectivity to HFO-1123, indicating lower activity of this catalyst for complete hydrogenation. In addition, the undesired byproduct formation is catalyst support dependent. It appears that R-152a formation follows alpha(α)>theta(θ)>delta(δ) trend. On the other hand, R-142 isomers' formation follows alpha(α)<theta(θ)<delta(δ) trend.

TABLE 17
Mole composition after a reactor packed with 0.2% Pd/alpha(α)-
Al₂O₃, 0.3% Pd/theta(θ)-Al₂O₃, and 0.3% Pd/delta(δ)-Al₂O₃ at 150 ± 1° C.

| | | | | | | mol. % (undesired) | | | | | | | |
|---|---|---|---|---|---|---|---|---|---|---|---|---|---|
| Catalyst | mol. % (desired) | | | | | | | | | R-142 | | | |
| support | R-143 | R-133b | R-133 | R-1123 | total | R-170 | R-143a | R-134a | R-152a | isomers | R-160 | Other * | total |
| alpha(α) | 84.61 | 1.26 | 1.59 | 0.75 | 88.21 | 1.59 | 0.86 | 0.02 | 3.60 | 0.33 | 0.88 | 4.51 | 11.79 |
| theta(θ) | 83.78 | 3.72 | 2.75 | 0.12 | 90.37 | 1.54 | 1.63 | 1.03 | 1.19 | 2.09 | 0.89 | 1.26 | 9.63 |
| delta(δ) | 72.48 | 4.86 | 2.47 | 9.92 | 89.73 | 0.63 | 0.38 | 0.23 | 0.76 | 5.93 | 0.7 | 1.61 | 10.27 |

Table 18 shows the product distribution for the first runs at 200° C. The 0.2% Pd/alpha(α)-Al₂O₃ catalyst showed the highest selectivity to R-143. The feed material was >99.99% pure CTFE, organic flow rate was 10 g/h, H₂ flow rate was 150 ml/min, and pressure was 45 psig. CTFE conversion was 100%. The 0.3% Pd/delta(δ)-Al₂O₃ catalyst showed 27.11% selectivity to HFO-1123, indicating lower activity of this catalyst for complete hydrogenation.

In addition, the undesired byproduct formation is catalyst support dependent. At 200° C., the major byproducts varied a lot depending on the catalyst support. The 0.2% Pd/alpha(α)-Al₂O₃ catalyst showed lowest and the 0.3% Pd/theta(θ)-Al₂O₃ catalyst showed the highest selectivity to undesired byproducts. For the 0.2% Pd/alpha(α)-Al₂O₃ catalyst, the major byproducts were R-170 (4.81%) and R-143a (4.42%). For the 0.3% Pd/theta(θ)-Al₂O₃ catalyst, the major byproducts were R-170 (12.33%) and R-134a (26.67%). For the 0.3% Pd/delta(δ)-Al₂O₃ catalyst, the major byproducts were R-170 (4.32%) and R-134a (14.68%). It appears that hydrogenation happens on all catalysts and 0.2% Pd/alpha-alumina showed the lowest activity to dehydrofluorination reactions.

TABLE 18
Mole composition after a reactor packed with 0.2% Pd/alpha(α)-
Al₂O₃, 0.3% Pd/theta(θ)-Al₂O₃, and 0.3% Pd/delta(δ)-Al₂O₃ at 200 ± 1° C.

| | | | | | | mol. % (undesired) | | | | | | | |
|---|---|---|---|---|---|---|---|---|---|---|---|---|---|
| Catalyst | mol. % (desired) | | | | | | | | | R-142 | | | |
| support | R-143 | R-133b | R-133 | R-1123 | total | R-170 | R-143a | R-134a | R-152a | isomers | R-160 | Other * | total |
| alpha(α) | 79.95 | 2.23 | 2.49 | 0.00 | 84.66 | 4.81 | 4.42 | 0.99 | 0.57 | 1.49 | 1.41 | 1.65 | 15.34 |
| theta(θ) | 44.08 | 4.60 | 4.64 | 0.00 | 53.33 | 12.33 | 2.67 | 26.67 | 0.02 | 2.19 | 1.32 | 1.47 | 46.67 |
| delta(δ) | 37.49 | 4.55 | 2.59 | 27.11 | 71.74 | 4.32 | 2.01 | 14.68 | 0.10 | 0.22 | 1.56 | 5.37 | 28.26 |

Table 19 shows the product distribution for the second runs at 150° C., after 200° C. (250° C. for the 0.2% Pd/alpha(α)-Al₂O₃ catalyst) runs. The 0.2% Pd/alpha(α)-Al₂O₃ catalyst again showed the highest selectivity to R-143. The feed material was >99.99% pure CTFE, organic flow rate was 10 g/h, H₂ flow rate was 150 ml/min, and pressure was 45 psig. CTFE conversion was 100%.

The 0.3% Pd/delta(δ)-Al₂O₃ catalyst showed 23.60% HFO-1123 formation, which is a lot more compared to the run at the same temperature on a fresh catalyst (9.92%). This catalyst showed significant deactivation after running the reaction at higher temperatures.

The 0.3% Pd/theta(θ)-Al₂O₃ catalyst showed slight increase in the formation of R-133 and R-133b intermediates compared to the run at the same temperature on a fresh catalyst (9.92%). This is consistent with reduced activity of the catalysts (deactivation) after high temperature runs.

In contrast to the two other catalysts, for the 0.2% Pd/alpha(α)-Al₂O₃ catalyst selectivity to R-143 increased after running the reaction at higher temperatures. This is consistent with higher stability of alpha(α)-Al₂O₃ and its lowest activity toward dehydrofluorination reaction compared to two other supports.

TABLE 19
Mole composition after a reactor packed with 0.2% Pd/alpha(α)-
Al₂O₃, 0.3% Pd/theta(θ)-Al₂O₃, and 0.3% Pd/delta(δ)-Al₂O₃ at 150 ± 1° C.

| | | | | | | mol. % (undesired) | | | | | | | |
|---|---|---|---|---|---|---|---|---|---|---|---|---|---|
| Catalyst | mol. % (desired) | | | | | | | | | R-142 | | | |
| support | R-143 | R-133b | R-133 | R-1123 | total | R-170 | R-143a | R-134a | R-152a | isomers | R-160 | Other * | total |
| alpha(α) | 87.86 | 3.65 | 2.16 | 0.17 | 93.83 | 1.01 | 1.50 | 0.47 | 2.14 | 0.19 | 0.45 | 0.40 | 6.17 |
| theta(θ) | 77.17 | 7.33 | 5.85 | 0.00 | 90.34 | 1.56 | 1.32 | 4.24 | 0.17 | 1.47 | 0.54 | 0.36 | 9.66 |
| delta(δ) | 65.95 | 4.60 | 1.77 | 23.60 | 95.93 | 0.26 | 0.96 | 1.42 | 0.41 | 0.55 | 0.25 | 0.22 | 4.07 |

As shown in Table 20, the highest selectivity to R-143, and the highest overall selectivity to R-143 and recyclable intermediates was obtained for the 0.2% Pd/alpha(α)-Al₂O₃ catalyst at 100° C. The feed material was >99.99% pure CTFE, organic flow rate was 10 g/h, H₂ flow rate was 150 ml/min, and pressure was 45 psig. CTFE conversion was 100%.

TABLE 20
Mole composition after a reactor packed with 0.2% Pd/alpha(α)-Al₂O₃,
0.3% Pd/theta(θ)-Al₂O₃, and 0.3% Pd/delta(δ)-Al₂O₃ at 100 to 130 ± 1° C.

| | | | | | | mol. % (undesired) | | | | | | | |
|---|---|---|---|---|---|---|---|---|---|---|---|---|---|
| Catalyst | mol. % (desired) | | | | | | | | | R-142 | | | |
| support | R-143 | R-133b | R-133 | R-1123 | total | R-170 | R-143a | R-134a | R-152a | isomers | R-160 | Other * | total |
| alpha(α) | 90.85 | 2.94 | 1.63 | 2.00 | 97.42 | 0.14 | 0.05 | 0.05 | 1.62 | 0.05 | 0.10 | 0.57 | 2.58 |
| (100° C.) | | | | | | | | | | | | | |
| alpha(α) | 88.94 | 3.58 | 2.03 | 0.25 | 94.80 | 0.86 | 1.51 | 0.41 | 1.57 | 0.13 | 0.45 | 0.27 | 5.20 |
| (130° C.) | | | | | | | | | | | | | |
| theta(θ) | 82.45 | 7.84 | 6.38 | 0.00 | 96.67 | 0.43 | 0.70 | 0.93 | 0.27 | 0.53 | 0.26 | 0.21 | 3.33 |
| (130° C.) | | | | | | | | | | | | | |
| delta(δ) | 62.94 | 3.68 | 1.40 | 30.21 | 98.24 | 0.08 | 0.48 | 0.44 | 0.48 | 0.09 | 0.09 | 0.10 | 1.76 |
| (130° C.) | | | | | | | | | | | | | |

### Example 5: CTFE and HFO-1123 as Feed Materials Using 0.3% Pd/Theta (θ)-Al₂O₃ Catalyst

This example shows conversion of chlorotrifluoroethylene (CTFE) and trifluoroethylene (HFO-1123 or TrFE) to HFC-143 using H₂ and 0.3% Pd/theta(θ)-Al₂O₃ catalyst. The surface area of this catalyst is 38.1 m²/g. The experimental apparatus for this example is same as Example 1. Organic flow rate is 10 g/h, H₂ flow rate is 150 ml/min, and pressure is 45 psig.

Prior to the reactions, the catalyst is pretreated under 150 ml/min of H₂ at 200° C. for four hours in the same reactor. Then, temperature is reduced to 130° C. under flowing hydrogen. When temperature is stable at 130° C., 10 g/h of a feed material comprising about 95 mol. % of CTFE and about 5 mol. % of HFO-1123 is added to the feed stream. Temperature typically increases about 15° C. to 20° C. upon introduction of the feed materials. For Example 5, the reactor temperature is adjusted to about 150° C. if it was different from 150° C. CTFE conversion is 100%.

The product stream comprises of R-143 (84.59%), R-133b (3.53%), R-133 (2.61%) and R-1123 (0.11%). The major byproducts are R-152a (1.13%), R-170 (1.46%), R-160 (0.85%), R-143a (1.55%), R-142 isomers (1.99%) and R-134a (0.98%).

### Example 6: CTFE and HFO-1123 as Feed Materials Using 0.2% Pd/Alpha (α)-Al₂O₃ Catalyst

This example shows conversion of chlorotrifluoroethylene (CTFE) and trifluoroethylene (HFO-1123 or TrFE) to HFC-143 using H₂ and 0.2% Pd/alpha(α)-Al₂O₃ catalyst. The surface area of this catalyst is 2.6 m²/g. The experimental apparatus for this example is same as Example 1. Organic flow rate is 10 g/h, H₂ flow rate is 150 ml/min, and pressure is 45 psig.

Prior to the reactions, the catalyst is subjected to the pretreatment and runs same as described in Example 1. The feed material comprising 95 mol. % CTFE and 5 mol. % HFO-1123 is used. CTFE conversions are 100% at both 100° C. and 150° C.

The product stream at 100° C. comprises of R-143 (91.31%), R-133b (2.79%), R-133 (1.55%) and R-1123 (1.90%). The major byproducts are R-152a (1.54%), R-170 (0.13%), R-160 (0.01%), R-143a (0.05%), R-142 isomers (0.05%) and R-134a (0.05%).

The product stream at 150° C. comprises of R-143 (88.47%), R-133b (3.47%), R-133 (2.05%) and R-1123 (0.16%). The major byproducts are R-152a (2.03%), R-170 (0.96%), R-160 (0.43%), R-143a (1.43%), R-142 isomers (0.18%) and R-134a (0.45%).

### Examples 7-50

### Step 2—Dehydrofluorination of 1,1,2-Trifluoroethane (HFC-143) to Produce Trans-1,2-Difluoroethylene (HFO-1132E) and Cis-1,2-Difluoroethylene (HFO-1132Z)

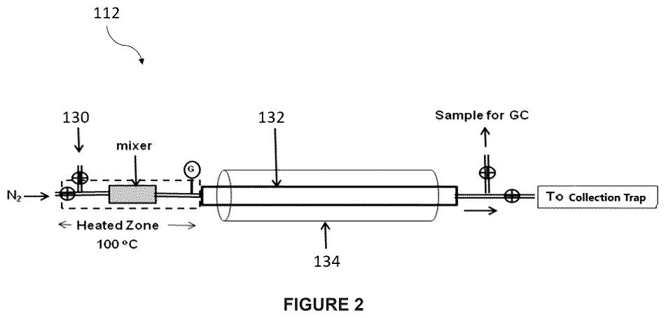

Referring to FIG. 2, the following Examples 7-50 were performed in reactor 112 according to the following procedure. A ½ inch Inconel tube reactor 132 was cleaned and flushed, then packed with a specified amount of catalyst, prepared as set forth below. The catalyst was then pretreated as set forth in Table 21 and 22 below and the tube reactor 132 was heated in an oven/furnace 134 at 250° C. for at least two hours while flowing nitrogen at atmospheric pressure to reach the foregoing temperature, followed by stopping the nitrogen flow. No oxygen or air was allowed to enter the reactor 112. Once the desired temperature was reached, the reactants were metered by a mass flow controller into the tube reactor 132 via reactant inlet 130 for approximately 1 hour at the desired pressure and flow rate. Nitrogen was metered into the tube to adjust the contact time in some cases. This step was followed by flowing the reactants through a DI water scrubber to remove HF and HCl. Aqueous 5% potassium hydroxide solution was also used to remove the HF and HCl from the reaction, no significant difference of 1132 ratio in the products was observed. Then the reaction products were collected in a cylinder chilled with dry ice followed by another cylinder chilled with liquid nitrogen. The products were analyzed by gas chromatography (GC), and the reaction selectivity and conversion was calculated.

### Preparation of Catalysts for Examples 7-50

### (I) Preparation of Fluorinated Chromium Oxide

Fluorinated chromium oxide was obtained via the procedure disclosed at col. 10, line 43 through col. 12, line 39 of U.S. Pat. No. 6,780,815 to Cerri et al. The fluorinated chromium oxide catalyst had a BET surface area of 50 m²/g. Before fluorination, the chromium oxide catalyst had a surface area of 211 m²/g.

### (II) Preparation of Fluorinated Alumina

300 g Alumina pellets 3×5 mm in a 1 inch Inconel tube reactor were slowly heated under 1 L/min N₂ to 200° C., held for 4 hours at 200° C., then ramped to 300° C. at 3° C./min heating rate. The catalyst was held for 4 hours at 300° C., then ramped to 400° C. at 3° C./min and held for 8 hours.

The catalyst was then cooled to 200° C., and N₂ was fed at 1 L/min at a pressure 20 psig, HF flow was initiated and fed into the tube reactor at ratio of 1 wt. % HF/N₂ mixture. The feeding rate was held until the catalyst temperature at all points was less than 215° C., before increasing the HF/N₂ ratio to 2.5 wt. %, 5 wt. %, 9.6 wt. %, 14 wt. %, 21 wt. %, and 25 wt. %, HF/N₂ ratio was increased to next level only after the catalyst temperature was stable or below 215° C. at all points. After reaching 25 wt. % of HF/N₂ ratio, the tube reactor temperature was slowly ramped to 350° C. at rate of 3° C./min, and this temperature was held for 2 hours while 25 wt. % HF/N₂ was continuously fed through the catalyst bed. The temperature was then ramped to 400° C. at rate of 3° C./min and held at 25 wt. % HF/N₂ for 2 hours, followed by increase of the pressure to 120 psig, and the flow of N₂ flow was stopped and switched to 100 wt. % HF at 120 psig and 1.5 lbs/h for 16 hours. The HF feed was then discontinued followed by re-starting N₂ flow at 6 L/min with cooling to room temperature.

### (III) Preparation of 1 wt. % Pd on Fluorinated Alumina (1% Pd on AlF₃)

For Examples 15-18, 1.25 g of Pd (NO₃)₂·2H₂O was dissolved in 100 ml DI water, and 50.0 g of AlF₃ pellets (5×3 mm from Johnson Matthey) were added into the solution. The resulted mixture was soaked overnight at room temperature, then water was removed under vacuum. The resulted solid was further dried at 150° C. under vacuum for 4 h to give 36.6 g pre-catalyst.

The pre-catalyst was then dried under 200 ml/min air flow at 400° C. for 4 hours, then reduced with 100 ml/min hydrogen flow at 400° C. for 2 h. The reduced catalyst was then used for the dehydrofluorination reactions below.

### (IV) Preparation of Fluorinated Magnesium Oxide

For Examples 19-22, 300 g of magnesium oxide chips (from MgO crystals) was placed in 1 inch Inconel tube and treated in the same manner as the fluorinated alumina in (II) above.

### (V) Preparation of 8.9 wt. % Ni on Fluorinated Alumina (1% Ni on AlF₃)

For Examples 23-24, 8.9% Ni on fluorinated alumina was prepared similarly, with 24.7 g of Ni(NO₃)₂·6H₂O dissolved in 100 ml DI water, and 51.3 g of AlF₃ pellets was added into the solution. The resulted solution was aged overnight, then water was removed under vacuum.

The resulted solid was further dried under vacuum at 150° C. for 4 h to give 51.5 g greyish solid. The pre-catalyst was further dried at 400° C. under 200 ml/min air flow for 4 hours, then reduced with 100 ml/min H₂ flow for 2 hours at 400° C. The reduced catalyst was then used for dehydrofluorination reactions below.

A summary of the reaction conditions is provided in Table 21 below:

TABLE 21
Reaction Conditions for Dehydrofluorination
of HFC-143 to Produce HFO-1132E and HFO-1132Z

| | | | | Reactor | Contact | | Catalyst |
|---|---|---|---|---|---|---|---|
| | Feed materials | Temp | Press | Volume | Time | | Pre- |
| Example | and feed rates | (° C.) | (psia) | (ml) | (sec) | Catalyst | treatment |
| 7 | 1,1,2- | 380 | 15 | 20.57 | 6.2 | Fluorinated | catalyst was |
| | trifluoroethane | | | | | chromium | slowly heated |
| | rate 80 ml/min, | | | | | oxide | to 250° C., |
| | N₂ rate 10 ml/min, | | | | | | then at rate |
| | | | | | | | of 3° C./min |
| | | | | | | | to 380° C. |
| 8 | 1,1,2- | 400 | 15 | 20.57 | 3.4 | catalyst was | catalyst was |
| | trifluoroethane | | | | | reused from | reused from |
| | rate 100 ml/min, | | | | | Ex. 7 | Ex. 7 |
| | N₂ rate 60 ml/min, | | | | | | |
| 9 | 1,1,2- | 350 | 15 | 20.57 | 3.1 | catalyst was | catalyst was |
| | trifluoroethane | | | | | reused from | reused from |
| | rate 8 g/h, N₂ | | | | | Ex. 8 | Ex. 8 |
| | rate 150 ml/min, | | | | | | |
| 10 | 1,1,2- | 380 | 15 | 20.57 | 6.3 | catalyst was | catalyst was |
| | trifluoroethane | | | | | reused from | reused from |
| | rate 6 g/h, N₂ | | | | | Ex. 9 | Ex. 9 |
| | rate 60 ml/min, | | | | | | |
| 11 | 1,1,2- | 380 | 15 | 20.57 | 3.1 | catalyst was | catalyst was |
| | trifluoroethane | | | | | reused from | oxidized with |
| | rate 8 g/h, N₂ | | | | | Ex. 10 | 200 ml/min air |
| | rate 140 ml/min, | | | | | | at 250-380° C. |
| | | | | | | | for 4 hours |
| | | | | | | | before use |
| 12 | 1,1,2- | 380 | 15 | 22.03 | 3.3 | Fluorinated | catalyst was |
| | trifluoroethane | | | | | alumina (gamma- | pretreated |
| | rate 8 g/h, N₂ | | | | | alumina pellets | at 500° C. |
| | rate 140 ml/min, | | | | | was treated with | for 4 hours |
| | | | | | | HF at 400° C., | before use |
| | | | | | | 120 Psig for 16 h) | |
| 13 | 1,1,2- | 350 | 15 | 22.03 | 3.3 | catalyst was | catalyst was |
| | trifluoroethane | | | | | reused from | reused from |
| | rate 8 g/h, N₂ | | | | | Ex. 12 | Ex. 12 |
| | rate 150 ml/min, | | | | | | |
| 14 | 1,1,2- | 380 | 15 | 22.03 | 1.1 | catalyst was | catalyst was |
| | trifluoroethane | | | | | reused from | reused from |
| | rate 8 g/h, N₂ | | | | | Ex. 13 | Ex. 13 |
| | rate 140 ml/min, | | | | | | |
| 15 | 1,1,2- | 380 | 15 | 22.03 | 1.1 | 1% Pd on | catalyst was |
| | trifluoroethane | | | | | AlF₃ pellets | pretreated |
| | rate 30 g/h, N₂ | | | | | (wet | at 400° C. |
| | rate 400 ml/min, | | | | | impregnated) | for 4 hours |
| | | | | | | | before use |
| 16 | 1,1,2- | 380 | 15 | 22.03 | 3.2 | catalyst was | catalyst was |
| | trifluoroethane | | | | | reused from | reused from |
| | rate 8 g/h, N₂ | | | | | Ex. 15 | Ex. 15 |
| | rate 150 ml/min, | | | | | | |
| 17 | 1,1,2- | 350 | 15 | 22.03 | 3.2 | catalyst was | catalyst was |
| | trifluoroethane | | | | | reused from | reused from |
| | rate 8 g/h, N₂ | | | | | Ex. 16 | Ex. 16 |
| | rate 160 ml/min, | | | | | | |
| 18 | 1,1,2- | 380 | 15 | 22.03 | 1.1 | catalyst was | catalyst was |
| | trifluoroethane | | | | | reused from | reused from |
| | rate 30 g/h, N₂ | | | | | Ex. 17 | Ex. 17 |
| | rate 400 ml/min, | | | | | | |
| 19 | 1,1,2- | 380 | 15 | 22.54 | 3.3 | Fluorinated | catalyst was |
| | trifluoroethane | | | | | magnesium | pretreated |
| | rate 5.5 g/h, N₂ | | | | | oxide (pellets | at 400° C. |
| | rate 160 ml/min, | | | | | was treated with | for 4 hours |
| | | | | | | HF at 400 C., | before use |
| | | | | | | 120 Psig for 16 h) | |
| 20 | 1,1,2- | 500 | 15 | 22.54 | 3.2 | catalyst was | catalyst was |
| | trifluoroethane | | | | | reused from | reused from |
| | rate 3 g/h, N₂ | | | | | Ex. 19 | Ex. 23 |
| | rate 145 ml/min, | | | | | | |
| 21 | 1,1,2- | 600 | 15 | 22.54 | 3.3 | catalyst was | catalyst was |
| | trifluoroethane | | | | | reused from | reused from |
| | rate 5 g/h, N₂ | | | | | Ex. 20 | Ex. 20 |
| | rate 115 ml/min, | | | | | | |
| 22 | 1,1,2- | 600 | 15 | 22.54 | 1.2 | catalyst was | catalyst was |
| | trifluoroethane | | | | | reused from | reused from |
| | rate 7.5 g/h, N₂ | | | | | Ex. 21 | Ex. 21 |
| | rate 350 ml/min, | | | | | | |
| 23 | 1,1,2- | 380 | 15 | 21.77 | 3.2 | 8.9% Ni on | catalyst |
| | trifluoroethane | | | | | AlF₃ pellets | Ni(NO₃)₂ |
| | rate 8 g/h, N₂ | | | | | (wet | on AlF₃ |
| | rate 140 ml/min, | | | | | impregnated) | (8.9% Ni) |
| | | | | | | | pellets was |
| | | | | | | | pretreated |
| | | | | | | | at 400° C. |
| | | | | | | | for 4 h with |
| | | | | | | | N₂, then |
| | | | | | | | reduced |
| | | | | | | | with 100% H₂ |
| | | | | | | | at 400° C. |
| | | | | | | | for 2 h |
| | | | | | | | before use |
| 24 | 1,1,2- | 380 | 15 | 21.77 | 1.2 | catalyst was | catalyst was |
| | trifluoroethane | | | | | reused from | reused from |
| | rate 5 g/h, N₂ | | | | | Ex. 23 | Ex. 23 |
| | rate 160 ml/min, | | | | | | |
| 25 | 1,1,2- | 300 | 15 | 2.57 | 3.31 | Fluorinated | catalyst was |
| | trifluoroethane | | | | | alumina (gamma- | pretreated |
| | rate 5.0 g/h | | | | | alumina pellets | at 400° C. |
| | | | | | | was treated with | for 4 h |
| | | | | | | HF at 400° C., | under N₂ flow |
| | | | | | | 120 Psig for 16 h) | before use |
| 26 | 1,1,2- | 320 | 15 | 2.57 | 3.26 | catalyst was | catalyst was |
| | trifluoroethane | | | | | reused from | reused from |
| | rate 4.9 g/h | | | | | Ex. 25 | Ex. 25 |
| 27 | 1,1,2- | 340 | 15 | 2.57 | 3.29 | catalyst was | catalyst was |
| | trifluoroethane | | | | | reused from | reused from |
| | rate 4.7 g/h | | | | | Ex. 26 | Ex. 26 |
| 28 | 1,1,2- | 340 | 15 | 2.57 | 3.29 | catalyst was | catalyst was |
| | trifluoroethane | | | | | reused from | reused from |
| | rate 4.7 g/h | | | | | Ex. 27 | Ex. 27 |
| 29 | 1,1,2- | 360 | 15 | 2.57 | 3.25 | catalyst was | catalyst was |
| | trifluoroethane | | | | | reused from | reused from |
| | rate 4.6 g/h | | | | | Ex. 28 | Ex. 28 |
| 30 | 1,1,2- | 360 | 15 | 2.57 | 1.25 | catalyst was | catalyst was |
| | trifluoroethane | | | | | reused from | reused from |
| | rate 12.0 g/h | | | | | Ex. 29 | Ex. 29 |
| 31 | 1,1,2- | 360 | 15 | 2.57 | 5.0 | catalyst was | catalyst was |
| | trifluoroethane | | | | | reused from | reused from |
| | rate 3.0 g/h | | | | | Ex. 30 | Ex. 30 |
| 32 | 1,1,2- | 340 | 15 | 2.57 | 4.99 | catalyst was | catalyst was |
| | trifluoroethane | | | | | reused from | reused from |
| | rate 3.1 g/h | | | | | Ex. 31 | Ex. 31 |
| 33 | 1,1,2- | 380 | 15 | 2.57 | 3.29 | catalyst was | catalyst was |
| | trifluoroethane | | | | | reused from | reused from |
| | rate 4.4 g/h | | | | | Ex. 32 | Ex. 32 |
| 34 | 1,1,2- | 380 | 15 | 2.57 | 1.21 | catalyst was | catalyst was |
| | trifluoroethane | | | | | reused from | reused from |
| | rate 12.0 g/h | | | | | Ex. 33 | Ex. 33 |
| 35 | 1,1,2- | 380 | 15 | 2.57 | 5.01 | catalyst was | catalyst was |
| | trifluoroethane | | | | | reused from | reused from |
| | rate 2.9 g/h | | | | | Ex. 34 | Ex. 34 |
| 36 | 1,1,2- | 360 | 15 | 2.57 | 3.26 | catalyst was | catalyst was |
| | trifluoroethane | | | | | reused from | reused from |
| | rate 4.6 g/h | | | | | Ex. 35 | Ex. 35 |
| 37 | 1,1,2- | 320 | 15 | 2.57 | 3.26 | Fluorinated | catalyst was |
| | trifluoroethane | | | | | chromium | slowly heated |
| | rate 4.9 g/h | | | | | oxide | to 250° C. |
| | | | | | | | under N₂, then |
| | | | | | | | at rate of |
| | | | | | | | 3° C./min |
| | | | | | | | to 350° C., |
| | | | | | | | and stayed at |
| | | | | | | | 350° C. for |
| | | | | | | | 4 h under |
| | | | | | | | N₂ flow |
| | | | | | | | before use |
| 38 | 1,1,2- | 320 | 15 | 2.57 | 3.26 | catalyst was | catalyst was |
| | trifluoroethane | | | | | reused from | reused from |
| | rate 4.9 g/h | | | | | Ex. 37 | Ex. 37 |
| 39 | 1,1,2- | 320 | 15 | 2.57 | 4.99 | catalyst was | catalyst was |
| | trifluoroethane | | | | | reused from | reused from |
| | rate 3.2 g/h | | | | | Ex. 38 | Ex. 38 |
| 40 | 1,1,2- | 340 | 15 | 2.57 | 3.29 | catalyst was | catalyst was |
| | trifluoroethane | | | | | reused from | reused from |
| | rate 4.7 g/h | | | | | Ex. 39 | Ex. 39 |
| 41 | 1,1,2- | 340 | 15 | 2.57 | 4.99 | catalyst was | catalyst was |
| | trifluoroethane | | | | | reused from | reused from |
| | rate 3.1 g/h | | | | | Ex. 40 | Ex. 40 |
| 42 | 1,1,2- | 360 | 15 | 2.57 | 4.99 | catalyst was | catalyst was |
| | trifluoroethane | | | | | reused from | reused from |
| | rate 3.0 g/h | | | | | Ex. 41 | Ex. 41 |
| 43 | 1,1,2- | 360 | 15 | 2.57 | 3.26 | catalyst was | catalyst was |
| | trifluoroethane | | | | | reused from | reused from |
| | rate 4.6 g/h | | | | | Ex. 42 | Ex. 42 |
| 44 | 1,1,2- | 360 | 15 | 2.57 | 1.25 | catalyst was | catalyst was |
| | trifluoroethane | | | | | reused from | reused from |
| | rate 12.0 g/h | | | | | Ex. 43 | Ex. 43 |
| 45 | 1,1,2- | 360 | 15 | 2.57 | 3.26 | Fluorinated | catalyst was |
| | trifluoroethane | | | | | chromium | slowly heated |
| | rate 4.6 g/h | | | | | oxide | to 250° C. |
| | | | | | | | under N₂, then |
| | | | | | | | at rate of |
| | | | | | | | 3° C./min |
| | | | | | | | to 350° C., |
| | | | | | | | and stayed at |
| | | | | | | | 350° C. for |
| | | | | | | | 4 h under |
| | | | | | | | N₂ flow |
| | | | | | | | before use |
| 46 | 1,1,2- | 360 | 15 | 2.57 | 1.25 | catalyst was | catalyst was |
| | trifluoroethane | | | | | reused from | reused from |
| | rate 12.0 g/h | | | | | Ex. 45 | Ex. 45 |
| 47 | 1,1,2- | 360 | 15 | 2.57 | 4.99 | catalyst was | catalyst was |
| | trifluoroethane | | | | | reused from | reused from |
| | rate 3.0 g/h | | | | | Ex. 46 | Ex. 46 |
| 48 | 1,1,2- | 380 | 15 | 2.57 | 3.3 | catalyst was | catalyst was |
| | trifluoroethane | | | | | reused from | reused from |
| | rate 4.4 g/h | | | | | Ex. 47 | Ex. 47 |
| 49 | 1,1,2- | 380 | 15 | 2.57 | 1.21 | catalyst was | catalyst was |
| | trifluoroethane | | | | | reused from | reused from |
| | rate 12.0 g/h | | | | | Ex. 48 | Ex. 48 |
| 50 | 1,1,2- | 380 | 15 | 2.57 | 5.01 | catalyst was | catalyst was |
| | trifluoroethane | | | | | reused from | reused from |
| | rate 2.9 g/h | | | | | Ex. 49 | Ex. 49 |

The reactions in Table 21 provided the product mixtures in Table 22 below.

TABLE 22
Summary of Products for Dehydrofluorination
of HFC-143 to Produce HFO-1132E and HFO-1132Z

| | | | | Selectivity to | Conversion |
|---|---|---|---|---|---|
| | | | Cis/trans | desired product | of starting |
| | Product Composition and | | molar | (HFO-1132E | material |
| Example | Amount (mol. %) | | ratio | and HFO-1132Z) | (HFC-143) |
| 7 | HFO-1132Z (cis-1,2- | 45.58 | 2.29 | 89.0% | 73.5% |
| | difluoroethylene) | | | | |
| | HFC-143 (1,1,2- | 26.49 | | | |
| | trifluoroethane) | | | | |
| | HFO-1132E (trans- | 19.87 | | | |
| | 1,2-difluoroethylene) | | | | |
| | HFC-143a (1,1,1- | 5.97 | | | |
| | trifluoroethane) | | | | |
| | Unidentified | 5.03 | | | |
| | components | | | | |
| 8 | HFO-1132Z (cis-1,2- | 54.08 | 3.68 | 95.6% | 71.9% |
| | difluoroethylene) | | | | |
| | HFC-143 (1,1,2- | 28.06 | | | |
| | trifluoroethane) | | | | |
| | HFO-1132E (trans- | 14.71 | | | |
| | 1,2-difluoroethylene) | | | | |
| | HFC-143a (1,1,1- | 2.20 | | | |
| | trifluoroethane) | | | | |
| | Unidentified | 2.20 | | | |
| | components | | | | |
| 9 | HFC-143 (1,1,2- | 56.23 | 9.4 | 96.0% | 43.8% |
| | trifluoroethane) | | | | |
| | HFO-1132Z (cis-1,2- | 38.0 | | | |
| | difluoroethylene) | | | | |
| | HFO-1132E (trans- | 4.04 | | | |
| | 1,2-difluoroethylene) | | | | |
| | HFC-143a (1,1,1- | 0.46 | | | |
| | trifluoroethane) | | | | |
| | Unidentified | 3.54 | | | |
| | components | | | | |
| 10 | HFO-1132Z (cis-1,2- | 53.23 | 4.66 | 95.9% | 67.4% |
| | difluoroethylene) | | | | |
| | HFC-143 (1,1,2- | 32.57 | | | |
| | trifluoroethane) | | | | |
| | HFO-1132E (trans- | 11.43 | | | |
| | 1,2-difluoroethylene) | | | | |
| | HFC-143a (1,1,1- | 1.39 | | | |
| | trifluoroethane) | | | | |
| | Unidentified | 2.71 | | | |
| | components | | | | |
| 11 | HFC-143 (1,1,2- | 56.23 | 2.85 | 95.5% | 85.2% |
| | trifluoroethane) | | | | |
| | HFO-1132Z (cis-1,2- | 38.0 | | | |
| | difluoroethylene) | | | | |
| | HFO-1132E (trans- | 4.04 | | | |
| | 1,2-difluoroethylene) | | | | |
| | HFC-143a (1,1,1- | 0.46 | | | |
| | trifluoroethane) | | | | |
| | Unidentified | 1.63 | | | |
| | components | | | | |
| 12 | HFO-1132Z (cis-1,2- | 49.39 | 2.51 | 96.3% | 71.2% |
| | difluoroethylene) | | | | |
| | HFC-143 (1,1,2- | 28.23 | | | |
| | trifluoroethane) | | | | |
| | HFO-1132E (trans- | 19.72 | | | |
| | 1,2-difluoroethylene) | | | | |
| | HFC-143a (1,1,1- | 1.82 | | | |
| | trifluoroethane) | | | | |
| | Unidentified | 0.84 | | | |
| | components | | | | |
| 13 | HFO-1132Z (cis-1,2- | 47.52 | 3.52 | 96.9% | 62.90% |
| | difluoroethylene) | | | | |
| | HFC-143 (1,1,2- | 37.03 | | | |
| | trifluoroethane) | | | | |
| | HFO-1132E (trans- | 13.5 | | | |
| | 1,2-difluoroethylene) | | | | |
| | HFC-143a (1,1,1- | 0.91 | | | |
| | trifluoroethane) | | | | |
| | Unidentified | 1.04 | | | |
| | components | | | | |
| 14 | HFO-1132Z (cis-1,2- | 53.04 | 4.91 | 97.7% | 65.30% |
| | difluoroethylene) | | | | |
| | HFC-143 (1,1,2- | 34.66 | | | |
| | trifluoroethane) | | | | |
| | HFO-1132E (trans- | 10.80 | | | |
| | 1,2-difluoroethylene) | | | | |
| | HFC-143a (1,1,1- | 0.54 | | | |
| | trifluoroethane) | | | | |
| | Unidentified | 0.96 | | | |
| | components | | | | |
| 15 | HFO-1132Z (cis-1,2- | 52.54 | 9.38 | 97.80% | 59.50% |
| | difluoroethylene) | | | | |
| | HFC-143 (1,1,2- | 40.53 | | | |
| | trifluoroethane) | | | | |
| | HFO-1132E (trans- | 5.6 | | | |
| | 1,2-difluoroethylene) | | | | |
| | HFC-143a (1,1,1- | 0.19 | | | |
| | trifluoroethane) | | | | |
| | Unidentified | 1.14 | | | |
| | components | | | | |
| 16 | HFO-1132Z (cis-1,2- | 57.29 | 4.53 | 98.00% | 71.40% |
| | difluoroethylene) | | | | |
| | HFC-143 (1,1,2- | 28.65 | | | |
| | trifluoroethane) | | | | |
| | HFO-1132E (trans- | 12.64 | | | |
| | 1,2-difluoroethylene) | | | | |
| | HFC-143a (1,1,1- | 0.53 | | | |
| | trifluoroethane) | | | | |
| | Unidentified | 0.89 | | | |
| | components | | | | |
| 17 | HFO-1132Z (cis-1,2- | 59.56 | 4.46 | 98.2% | 74.4% |
| | difluoroethylene) | | | | |
| | HFC-143 (1,1,2- | 25.77 | | | |
| | trifluoroethane) | | | | |
| | HFO-1132E (trans- | 13.35 | | | |
| | 1,2-difluoroethylene) | | | | |
| | HFC-143a (1,1,1- | 0.55 | | | |
| | trifluoroethane) | | | | |
| | Unidentified | 0.77 | | | |
| | components | | | | |
| 18 | HFO-1132Z (cis-1,2- | 62.18 | 6.22 | 98.60% | 73.20% |
| | difluoroethylene) | | | | |
| | HFC-143 (1,1,2- | 26.81 | | | |
| | trifluoroethane) | | | | |
| | HFO-1132E (trans- | 10.00 | | | |
| | 1,2-difluoroethylene) | | | | |
| | HFC-143a (1,1,1- | 0.35 | | | |
| | trifluoroethane) | | | | |
| | Unidentified | 0.66 | | | |
| | components | | | | |
| 19 | HFC-143 (1,1,2- | 95.8 | NA | 58.30% | 4.20% |
| | trifluoroethane) | | | | |
| | HFO-1132Z (cis-1,2- | 2.3 | | | |
| | difluoroethylene) | | | | |
| | HFO-1132E (trans- | 0 | | | |
| | 1,2-difluoroethylene) | | | | |
| | HFC-143a (1,1,1- | 0 | | | |
| | trifluoroethane) | | | | |
| | Unidentified | 1.84 | | | |
| | components | | | | |
| 20 | HFC-143 (1,1,2- | 74.85 | 13.9 | 94.10% | 25.20% |
| | trifluoroethane) | | | | |
| | HFO-1132Z (cis-1,2- | 22.07 | | | |
| | difluoroethylene) | | | | |
| | HFO-1132E (trans- | 1.59 | | | |
| | 1,2-difluoroethylene) | | | | |
| | HFC-143a (1,1,1- | 0.0 | | | |
| | trifluoroethane) | | | | |
| | Unidentified | 1.49 | | | |
| | components | | | | |
| 21 | HFO-1132Z (cis-1,2- | 54.24 | 8.6 | 97.90% | 61.80% |
| | difluoroethylene) | | | | |
| | HFC-143 (1,1,2- | 38.17 | | | |
| | trifluoroethane) | | | | |
| | HFO-1132E (trans- | 6.30 | | | |
| | 1,2-difluoroethylene) | | | | |
| | HFC-143a (1,1,1- | 0.25 | | | |
| | trifluoroethane) | | | | |
| | Unidentified | 1.04 | | | |
| | components | | | | |
| 22 | HFC-143 (1,1,2- | 71.46 | 9.5 | 94.80% | 28.50% |
| | trifluoroethane) | | | | |
| | HFO-1132Z (cis-1,2- | 24.49 | | | |
| | difluoroethylene) | | | | |
| | HFO-1132E (trans- | 2.57 | | | |
| | 1,2-difluoroethylene) | | | | |
| | HFC-143a (1,1,1- | 0.20 | | | |
| | trifluoroethane) | | | | |
| | Unidentified | 1.28 | | | |
| | components | | | | |
| 23 | HFO-1132Z (cis-1,2- | 70.56 | 3.2 | 97.8% | 94.6% |
| | difluoroethylene) | | | | |
| | HFO-1132E (trans- | 21.95 | | | |
| | 1,2-difluoroethylene) | | | | |
| | HFC-143 (1,1,2- | 5.37 | | | |
| | trifluoroethane) | | | | |
| | HFC-143a (1,1,1- | 1.05 | | | |
| | trifluoroethane) | | | | |
| | Unidentified | 1.07 | | | |
| | components | | | | |
| 24 | HFO-1132Z (cis-1,2- | 73.23 | 4.5 | 98.20% | 90.90% |
| | difluoroethylene) | | | | |
| | HFO-1132E (trans- | 16.12 | | | |
| | 1,2-difluoroethylene) | | | | |
| | HFC-143 (1,1,2- | 9.05 | | | |
| | trifluoroethane) | | | | |
| | HFC-143a (1,1,1- | 0.63 | | | |
| | trifluoroethane) | | | | |
| | Unidentified | 0.44 | | | |
| | components | | | | |
| 25 | HFC-143a (1,1,1- | 0.07 | 11.52 | 94.00% | 25.31% |
| | trifluoroethane) | | | | |
| | HFO-1132E (trans- | 1.9 | | | |
| | 1,2-difluoroethylene) | | | | |
| | HFO-1132Z (cis-1,2- | 21.88 | | | |
| | difluoroethylene) | | | | |
| | HFC-143 (1,1,2- | 74.69 | | | |
| | trifluoroethane) | | | | |
| | Unidentified | 1.46 | | | |
| | components | | | | |
| 26 | HFC-143a (1,1,1- | 0.10 | 11.52 | 95.03% | 32.58% |
| | trifluoroethane) | | | | |
| | HFO-1132E (trans- | 2.48 | | | |
| | 1,2-difluoroethylene) | | | | |
| | HFO-1132Z (cis-1,2- | 28.57 | | | |
| | difluoroethylene) | | | | |
| | HFC-143 (1,1,2- | 67.42 | | | |
| | trifluoroethane) | | | | |
| | Unidentified | 1.43 | | | |
| | components | | | | |
| 27 | HFC-143a (1,1,1- | 0.06 | 15.61 | 94.89% | 28.19% |
| | trifluoroethane) | | | | |
| | HFO (fluoroethylene) | 0.03 | | | |
| | HFO-1132E (trans- | 1.61 | | | |
| | 1,2-difluoroethylene) | | | | |
| | HFO-1132Z (cis-1,2- | 25.14 | | | |
| | difluoroethylene) | | | | |
| | HFC-143 (1,1,2- | 71.81 | | | |
| | trifluoroethane) | | | | |
| | Unidentified | 1.35 | | | |
| | components | | | | |
| 28 | HFC-143a (1,1,1- | 0.10 | 13.49 | 95.90% | 34.94% |
| | trifluoroethane) | | | | |
| | HFO (fluoroethylene) | 0.04 | | | |
| | HFO-1132E (trans- | 2.31 | | | |
| | 1,2-difluoroethylene) | | | | |
| | HFO-1132Z (cis-1,2- | 31.18 | | | |
| | difluoroethylene) | | | | |
| | HFC-143 (1,1,2- | 65.06 | | | |
| | trifluoroethane) | | | | |
| | Unidentified | 1.31 | | | |
| | components | | | | |
| 29 | HFC-143a (1,1,1- | 0.117 | 12.62 | 96.20% | 33.14% |
| | trifluoroethane) | | | | |
| | HFO (fluoroethylene) | 0.02 | | | |
| | HFO-1132E (trans- | 2.73 | | | |
| | 1,2-difluoroethylene) | | | | |
| | HFO-1132Z (cis-1,2- | 34.44 | | | |
| | difluoroethylene) | | | | |
| | HFC-143 (1,1,2- | 61.29 | | | |
| | trifluoroethane) | | | | |
| | Unidentified | 1.40 | | | |
| | components | | | | |
| 30 | HFC-143a (1,1,1- | 0.03 | 13.99 | 92.80% | 19.23% |
| | trifluoroethane) | | | | |
| | HFO (fluoroethylene) | 0.03 | | | |
| | HFO-1132E (trans- | 1.19 | | | |
| | 1,2-difluoroethylene) | | | | |
| | HFO-1132Z (cis-1,2- | 16.65 | | | |
| | difluoroethylene) | | | | |
| | HFC-143 (1,1,2- | 80.77 | | | |
| | trifluoroethane) | | | | |
| | Unidentified | 1.33 | | | |
| | components | | | | |
| 31 | HFC-143a (1,1,1- | 0.11 | 14.24 | 95.90% | 40.83% |
| | trifluoroethane) | | | | |
| | HFO (fluoroethylene) | 0.05 | | | |
| | HFO-1132E (trans- | 2.57 | | | |
| | 1,2-difluoroethylene) | | | | |
| | HFO-1132Z (cis-1,2- | 36.6 | | | |
| | difluoroethylene) | | | | |
| | HFC-143 (1,1,2- | 59.17 | | | |
| | trifluoroethane) | | | | |
| | Unidentified | 1.50 | | | |
| | components | | | | |
| 32 | HFC-143a (1,1,1- | 0.03 | 15.92 | 95.30% | 21.29% |
| | trifluoroethane) | | | | |
| | HFO (fluoroethylene) | 0.06 | | | |
| | HFO-1132E (trans- | 1.2 | | | |
| | 1,2-difluoroethylene) | | | | |
| | HFO-1132Z (cis-1,2- | 19.1 | | | |
| | difluoroethylene) | | | | |
| | HFC-143 (1,1,2- | 78.71 | | | |
| | trifluoroethane) | | | | |
| | Unidentified | 1.51 | | | |
| | components | | | | |
| 33 | HFC-143a (1,1,1- | 0.17 | 11.32 | 96.90% | 48.28% |
| | trifluoroethane) | | | | |
| | HFO (fluoroethylene) | 0.08 | | | |
| | HFO-1132E (trans- | 3.8 | | | |
| | 1,2-difluoroethylene) | | | | |
| | HFO-1132Z (cis-1,2- | 43.0 | | | |
| | difluoroethylene) | | | | |
| | HFC-143 (1,1,2- | 51.72 | | | |
| | trifluoroethane) | | | | |
| | Unidentified | 1.23 | | | |
| | components | | | | |
| 34 | HFC-143a (1,1,1- | 0.04 | 14.9 | 92.90% | 22.24% |
| | trifluoroethane)ea | | | | |
| | HFO (fluoroethylene) | 0.03 | | | |
| | HFO-1132E (trans- | 1.2 | | | |
| | 1,2-difluoroethylene) | | | | |
| | HFO-1132Z (cis-1,2- | 19.37 | | | |
| | difluoroethylene) | | | | |
| | HFC-143 (1,1,2- | 77.76 | | | |
| | trifluoroethane) | | | | |
| | Unidentified | 1.50 | | | |
| | components | | | | |
| 35 | HFC-143a (1,1,1- | 0.19 | 11.66 | 96.70% | 50.51% |
| | trifluoroethane) | | | | |
| | HFO (fluoroethylene) | 0.08 | | | |
| | HFO-1132E (trans- | 3.86 | | | |
| | 1,2-difluoroethylene) | | | | |
| | HFO-1132Z (cis-1,2- | 44.99 | | | |
| | difluoroethylene) | | | | |
| | HFC-143 (1,1,2- | 49.49 | | | |
| | trifluoroethane) | | | | |
| | Unidentified | 1.39 | | | |
| | components | | | | |
| 36 | HFC-143a (1,1,1- | 0.14 | 11.47 | 96.95% | 41.94% |
| | trifluoroethane) | | | | |
| | HFO (fluoroethylene) | 0.06 | | | |
| | HFO-1132E (trans- | 3.26 | | | |
| | 1,2-difluoroethylene) | | | | |
| | HFO-1132Z (cis-1,2- | 37.40 | | | |
| | difluoroethylene) | | | | |
| | HFC-143 (1,1,2- | 58.06 | | | |
| | trifluoroethane) | | | | |
| | Unidentified | 1.80 | | | |
| | components | | | | |
| 37 | HFC-143a (1,1,1- | 0.95 | 6.38 | 94.40% | 41.70% |
| | trifluoroethane), | | | | |
| | HFO (fluoroethylene) | 0.07 | | | |
| | HFO-1132E (trans- | 5.33 | | | |
| | 1,2-difluoroethylene) | | | | |
| | HFO-1132Z (cis-1,2- | 34.03 | | | |
| | difluoroethylene) | | | | |
| | HFC-143 (1,1,2- | 58.30 | | | |
| | trifluoroethane) | | | | |
| | Unidentified | 1.32 | | | |
| | components | | | | |
| 38 | HFC-143a (1,1,1- | 0.50 | 8.04 | 94.80% | 35.08% |
| | trifluoroethane) | | | | |
| | HFO-1132E (trans- | 3.68 | | | |
| | 1,2-difluoroethylene) | | | | |
| | HFO-1132Z (cis-1,2- | 29.57 | | | |
| | difluoroethylene) | | | | |
| | HFC-143 (1,1,2- | 64.92 | | | |
| | trifluoroethane) | | | | |
| | Unidentified | 1.33 | | | |
| | components | | | | |
| 39 | HFC-143a (1,1,1- | 0.43 | 8.7 | 94.90% | 34.82% |
| | trifluoroethane) | | | | |
| | HFO-1132E (trans- | 3.41 | | | |
| | 1,2-difluoroethylene) | | | | |
| | HFO-1132Z (cis-1,2- | 29.65 | | | |
| | difluoroethylene) | | | | |
| | HFC-143 (1,1,2- | 65.18 | | | |
| | trifluoroethane) | | | | |
| | Unidentified | 1.40 | | | |
| | components | | | | |
| 40 | HFC-143a (1,1,1- | 0.47 | 7 | 95.50% | 37.25% |
| | trifluoroethane) | | | | |
| | HFO (fluoroethylene) | 0.03 | | | |
| | HFO-1132E (trans- | 4.45 | | | |
| | 1,2-difluoroethylene) | | | | |
| | HFO-1132Z (cis-1,2- | 31.14 | | | |
| | difluoroethylene) | | | | |
| | HFC-143 (1,1,2- | 62.75 | | | |
| | trifluoroethane) | | | | |
| | Unidentified | 1.16 | | | |
| | components | | | | |
| 41 | HFC-143a (1,1,1- | 0.33 | 8.73 | 95.40% | 34.80% |
| | trifluoroethane) | | | | |
| | HFO (fluoroethylene) | 0.03 | | | |
| | HFO-1132E (trans- | 3.41 | | | |
| | 1,2-difluoroethylene) | | | | |
| | HFO-1132Z (cis-1,2- | 28.79 | | | |
| | difluoroethylene) | | | | |
| | HFC-143 (1,1,2- | 65.20 | | | |
| | trifluoroethane) | | | | |
| | Unidentified | 1.25 | | | |
| | components | | | | |
| 42 | HFC-143a (1,1,1- | 0.55 | 6.23 | 96.10% | 44.58% |
| | trifluoroethane) | | | | |
| | HFO (fluoroethylene) | 0.06 | | | |
| | HFO-1132E (trans- | 5.93 | | | |
| | 1,2-difluoroethylene) | | | | |
| | HFO-1132Z (cis-1,2- | 36.92 | | | |
| | difluoroethylene) | | | | |
| | HFC-143 (1,1,2- | 55.42 | | | |
| | trifluoroethane) | | | | |
| | Unidentified | 1.12 | | | |
| | components | | | | |
| 43 | HFC-143a (1,1,1- | 0.18 | 9.09 | 93.50% | 25.16% |
| | trifluoroethane) | | | | |
| | HFO (fluoroethylene) | 0.03 | | | |
| | HFO-1132E (trans- | 2.33 | | | |
| | 1,2-difluoroethylene) | | | | |
| | HFO-1132Z (cis-1,2- | 21.19 | | | |
| | difluoroethylene) | | | | |
| | HFC-143 (1,1,2- | 74.84 | | | |
| | trifluoroethane) | | | | |
| | Unidentified | 1.43 | | | |
| | components | | | | |
| 44 | HFC-143a (1,1,1- | 0.06 | 9.33 | 87.40% | 11.93% |
| | trifluoroethane) | | | | |
| | HFO (fluoroethylene) | 0.02 | | | |
| | HFO-1132E (trans- | 1.01 | | | |
| | 1,2-difluoroethylene) | | | | |
| | HFO-1132Z (cis-1,2- | 9.42 | | | |
| | difluoroethylene) | | | | |
| | HFC-143 (1,1,2- | 88.07 | | | |
| | trifluoroethane) | | | | |
| | Unidentified | 1.42 | | | |
| | components | | | | |
| 45 | HFC-143a (1,1,1- | 2.38 | 4.41 | 93.30% | 58.61% |
| | trifluoroethane) | | | | (70% |
| | HFO-1132E (trans- | 10.11 | | | material |
| | 1,2-diHFO | | | | balance) |
| | (fluoroethylene)) | | | | |
| | HFO-1132Z (cis-1,2- | 44.59 | | | |
| | difluoroethylene) | | | | |
| | HFC-143 (1,1,2- | 41.39 | | | |
| | trifluoroethane) | | | | |
| | Unidentified | 1.53 | | | |
| | components | | | | |
| 46 | HFC-143a (1,1,1- | 0.27 | 7.94 | 91.40% | 21.92% |
| | trifluoroethane) | | | | (75% |
| | HFO-1132E (trans- | 2.24 | | | material |
| | 1,2-difluoroethylene) | | | | balance) |
| | HFO-1132Z (cis-1,2- | 17.79 | | | |
| | difluoroethylene) | | | | |
| | HFC-143 (1,1,2- | 78.08 | | | |
| | trifluoroethane) | | | | |
| | Unidentified | 1.62 | | | |
| | components | | | | |
| 47 | HFC-143a (1,1,1- | 1.73 | 3.97 | 95.90% | 67.90% |
| | trifluoroethane) | | | | (73% |
| | HFO (fluoroethylene) | 0.07 | | | material |
| | HFO-1132E (trans- | 13.11 | | | balance) |
| | 1,2-difluoroethylene) | | | | |
| | HFO-1132Z (cis-1,2- | 52.03 | | | |
| | difluoroethylene) | | | | |
| | HFC-143 (1,1,2- | 32.08 | | | |
| | trifluoroethane) | | | | |
| | Unidentified | 0.98 | | | |
| | components | | | | |
| 48 | HFC-143a (1,1,1- | 0.53 | 6.64 | 94.90% | 36.46% |
| | trifluoroethane) | | | | (91% |
| | HFO-1132E (trans- | 4.53 | | | material |
| | 1,2-difluoroethylene) | | | | balance) |
| | HFO-1132Z (cis-1,2- | 30.08 | | | |
| | difluoroethylene) | | | | |
| | HFC-143 (1,1,2- | 63.54 | | | |
| | trifluoroethane) | | | | |
| | Unidentified | 1.32 | | | |
| | components | | | | |
| 49 | HFC-143a (1,1,1- | 0.42 | 6.1 | 93.80% | 26.94% |
| | trifluoroethane) | | | | (91% |
| | HFO-1132E (trans- | 3.56 | | | material |
| | 1,2-difluoroethylene) | | | | balance) |
| | HFO-1132Z (cis-1,2- | 21.71 | | | |
| | difluoroethylene) | | | | |
| | HFC-143 (1,1,2- | 73.06 | | | |
| | trifluoroethane) | | | | |
| | Unidentified | 1.25 | | | |
| | components | | | | |
| 50 | HFC-143a (1,1,1- | 0.53 | 7.19 | 95.40% | 40.77% |
| | trifluoroethane) | | | | (73% |
| | HFO-1132E (trans- | 4.75 | | | material |
| | 1,2-difluoroethylene) | | | | balance) |
| | HFO-1132Z (cis-1,2- | 34.16 | | | |
| | difluoroethylene) | | | | |
| | HFC-143 (1,1,2- | 59.23 | | | |
| | trifluoroethane) | | | | |
| | Unidentified | 1.33 | | | |
| | components | | | | |

### Examples 51-92: Step (i)—Hydrogenation of Chlorotrifluoroethylene (CTFE) and/or HFO-1123 to Produce 1,1,2-Trifluoroethane (HFC-143) Using Different Catalysts

Examples 1-6 are repeated using the same experimental apparatus under the same conditions but with different catalysts. Prior to the reactions, the catalyst is subjected to the pretreatment and runs same as described in Examples 1-6. The feed material containing CTFE and/or HFO-1123 is converted to 1,1,2-trifluoroethane (HFC-143) in the presence of a catalyst and support listed in Table 23. Conversions of the feed materials are 100% at 100° C. and 150° C. Similar selectivity towards the end product and desired intermediates are achieved as results in Examples 1-6.

TABLE 23
Catalysts used for Hydrogenation of chlorotrifluoroethylene
(CTFE) to produce 1.1.2-trifluoroethane (HFC-143)

| | | | Catalyst |
|---|---|---|---|
| Example | Feed Material | Catalyst-Support | Loading |
| 51 | 99.99% pure CTFE | Pt/alpha(α)-Al₂O₃ | 0.1-10 wt. % |
| 52 | | Rh/alpha(α)-Al₂O₃ | |
| 53 | | Ru/alpha(α)-Al₂O₃ | |
| 54 | | Ir/alpha(α)-Al₂O₃ | |
| 55 | | Fe/alpha(α)-Al₂O₃ | 10-80 wt. % |
| 56 | | Co/alpha(α)-Al₂O₃ | |
| 57 | | Ni/alpha(α)-Al₂O₃ | |
| 58 | | Pt/theta(α)-Al₂O₃ | 0.1-10 wt. % |
| 59 | | Rh/theta(α)-Al₂O₃ | |
| 60 | | Ru/theta(α)-Al₂O₃ | |
| 61 | | Ir/alpha(α)-Al₂O₃ | |
| 62 | | Fe/theta(α)-Al₂O₃ | 10-80 wt. % |
| 63 | | Co/theta(α)-Al₂O₃ | |
| 64 | | Ni/theta(α)-Al₂O₃ | |
| 65 | 95 mol. % of CTFE | Pt/alpha(α)-Al₂O₃ | 0.1-10 wt. % |
| 66 | and 5 mol. % of HFO- | Rh/alpha(α)-Al₂O₃ | |
| 67 | 1123 | Ru/alpha(α)-Al₂O₃ | |
| 68 | | Ir/alpha(α)-Al₂O₃ | |
| 69 | | Fe/alpha(α)-Al₂O₃ | 10-80 wt. % |
| 70 | | Co/alpha(α)-Al₂O₃ | |
| 71 | | Ni/alpha(α)-Al₂O₃ | |
| 72 | | Pt/theta(α)-Al₂O₃ | 0.1-10 wt. % |
| 73 | | Rh/theta(α)-Al₂O₃ | |
| 74 | | Ru/theta(α)-Al₂O₃ | |
| 75 | | Ir/alpha(α)-Al₂O₃ | |
| 76 | | Fe/theta(α)-Al₂O₃ | 10-80 wt. % |
| 77 | | Co/theta(α)-Al₂O₃ | |
| 78 | | Ni/theta(α)-Al₂O₃ | |
| 79 | 50 mol. % of CTFE | Pt/alpha(α)-Al₂O₃ | 0.1-10 wt. % |
| 80 | and 50 mol. % of | Rh/alpha(α)-Al₂O₃ | |
| 81 | HFO-1123 | Ru/alpha(α)-Al₂O₃ | |
| 82 | | Ir/alpha(α)-Al₂O₃ | |
| 83 | | Fe/alpha(α)-Al₂O₃ | 10-80 wt. % |
| 84 | | Co/alpha(α)-Al₂O₃ | |
| 85 | | Ni/alpha(α)-Al₂O₃ | |
| 86 | | Pt/theta(α)-Al₂O₃ | 0.1-10 wt. % |
| 87 | | Rh/theta(α)-Al₂O₃ | |
| 88 | | Ru/theta(α)-Al₂O₃ | |
| 89 | | Ir/alpha(α)-Al₂O₃ | |
| 90 | | Fe/theta(α)-Al₂O₃ | 10-80 wt. % |
| 91 | | Co/theta(α)-Al₂O₃ | |
| 92 | | Ni/theta(α)-Al₂O₃ | |

### Example 93: CTFE and HFO-1123 as Feed Materials Using 0.3% Pd/Theta (8)-Al₂O₃Catalyst

This example shows conversion of chlorotrifluoroethylene (CTFE) and trifluoroethylene (HFO-1123 or TrFE) to HFC-143 using H₂ and 0.3% Pd/theta(θ)-Al₂O₃ catalyst. The surface area of this catalyst is 38.1 m²/g. The experimental apparatus for this example is same as Example 1. Organic flow rate is 10 g/h, H₂ flow rate is 150 ml/min, and pressure is 45 psig.

Prior to the reactions, the catalyst is pretreated under 150 ml/min of H₂ at 200° C. for four hours in the same reactor. Then, temperature is reduced to 130° C. under flowing hydrogen. When temperature is stable at 130° C., 10 g/h of a feed material comprising about 50 mol. % of CTFE and about 50 mol. % of HFO-1123 are added to the feed stream. Temperature typically increases about 15° C. to 20° C. upon introduction of the feed materials. For Example 87, the reactor temperature is adjusted to about 150° C. if different from 150° C. CTFE conversion is 100%.

The product stream comprises of R-143 (91.89%), R-133b (1.86%), R-133 (1.38%) and R-1123 (0.06%). The major byproducts were R-152a (0.60%), R-170 (0.77%), R-160 (0.45%), R-143a (0.82%), R-142 isomers (1.05%) and R-134a (0.52%).

### Example 94: CTFE and HFO-1123 as Feed Materials Using 0.2% Pd/Alpha (α)-Al₂O₃ Catalyst

This example shows conversion of chlorotrifluoroethylene (CTFE) and trifluoroethylene (HFO-1123 or TrFE) to HFC-143 using H₂ and 0.2% Pd/alpha(α)-Al₂O₃ catalyst. The surface area of this catalyst is 2.6 m²/g. The experimental apparatus for this example is same as Example 1. Organic flow rate is 10 g/h, H₂ flow rate is 150 ml/min, and pressure is 45 psig.

Prior to the reactions, the catalyst is subjected to the pretreatment and runs same as described in Example 1. The feed material comprising 50 mol. % CTFE and 50 mol. % HFO-1123 is used. Temperature typically increases about 15° C. to 20° C. upon introduction of the feed materials. For Example 88, the reactor temperature is adjusted to about 150° C. if it was different from 150° C. CTFE conversion is 100%.

The product stream comprises of R-143 (93.93%), R-133b (1.83%), R-133 (1.08%) and R-1123 (0.09%). The major byproducts were R-152a (1.07%), R-170 (0.51%), R-160 (0.23%), R-143a (0.75%), R-142 isomers (0.10%) and R-134a (0.24%).

### Example 95: Step (iii)—Isomerization of Cis-1,2-Difluoroethylene (HFO-1132Z) Isomer Converted to Trans-1,2-Difluoroethylene (HFO-1132E)

A mixture of predominantly cis-1,2-difluoroethylene (HFO-1132Z) isomer and a lesser amount of trans-1,2-difluoroethylene (HFO-1132E) is converted to substantially pure trans-1,2-difluoroethylene (HFO-1132E) isomer by exposure to heat and a catalyst.

The isomerization reaction is conducted in a vapor phase reactor resistant to corrosion such as nickel and its alloys, including Hastelloy (for example, Hastelloy C276), Inconel (for example Inconel 600), and Monel. The reaction is conducted at about 200° C. under vacuum in an inert atmosphere substantially devoid of oxygen.

Optionally, a distillation column is used to separate unconverted raw materials for recycle and target intermediates, byproducts, or products.

### Example 96: Step (i)—Hydrogenation of Chlorotrifluoroethylene (CTFE) to Produce 1,1,2-Trifluoroethane (HFC-143) Using 4% Pd/C Catalyst

The reaction apparatus was same as Example 1. For this example, a 1.5-inch SS tubular reactor with about 18 inches of length was filled with 500 ml of the neat 4% Pd/C catalyst. The catalyst was sandwiched between SS metal mesh on both sides of the reactor. A multipoint thermocouple (OD= 3/16 inch) was placed inside the reactor with a reading point every two inches. Note that the first and last thermocouple reading points were inside the mesh before/after the catalyst bed. The reactor was heated externally using a box oven.

Prior to the reaction, the catalyst was pretreated at 280° C. under 1000 ml/min of hydrogen at 0 psig for 8 hours. The oven temperature was reduced to 200° C., pressure adjusted to 45 psig, hydrogen flow rate reduced to 700 ml/min, and then organic was introduced at 50 g/h organic flow rate. The flow direction was from bottom to the top.

Table 24 shows the temperature profiles inside the reactor during the reaction. The oven temperature was about 200° C. while the hot spot temperature was about 293° C. Note that reaction happened only at the first few inches at the bottom of the reactor (flow direction was from bottom to top) while the rest of the reactor remained almost same temperature as the oven temperature. Table 25 shows the reactor effluent composition.

TABLE 24
Temperature profile inside the reactor

| Distance from the | |
|---|---|
| bottom of the reactor | |
| (inches) | Temperature (° C.) |
| 18 | 198 |
| 16 | 199 |
| 14 | 199 |
| 12 | 199 |
| 10 | 201 |
| 8 | 207 |
| 6 | 224 |
| 4 | 275 |
| 2 | 293 |
| 0 | 215 |

TABLE 25
Reactor effluent composition, wherein the presented data are average of 5
GC samples, taken every four hours from the reactor effluent over 17 hours

| | | | | | mol. % (undesired) | | | | | | | |
|---|---|---|---|---|---|---|---|---|---|---|---|---|
| mol. % (desired) | | | | | | | | | R-142 | | | |
| R-143 | R-133b | R-133 | R-1123 | total | R-170 | R-143a | R-134a | R-152a | isomers | R-160 | Others | total |
| 93.61 | 0.81 | 1.30 | 0.12 | 95.84 | 0.66 | 0.09 | 0.00 | 2.06 | 0.47 | 0.43 | 0.45 | 4.16 |

### ASPECTS

Aspect 1 is a method for producing HFC-143, comprising: reacting a reactant composition comprising chlorotrifluoroethylene (CTFE) with hydrogen in the presence of a catalyst at a temperature of between about 75° C. and about 225° C. to produce a product mixture comprising 1,1,2-trifluoroethane (HFC-143).

Aspect 2 is the method of Aspect 1, wherein the temperature is between about 100° C. and about 200° C.

Aspect 3 is the method of Aspect 1 or Aspect 2, wherein the temperature is between about 120° C. and about 180° C.

Aspect 4 is the method of any one of Aspects 1-3, wherein the reactant composition further comprises trifluoroethylene (HFO-1123).

Aspect 5 is the method of Aspect 4, wherein the reactant composition comprises: from about 50 mol. % to about 99.99 mol. % of CTFE; and from 0.01 mol. % to about 50 mol. % of HFO-1123, based on total moles of chlorotrifluoroethylene (CTFE) and trifluoroethylene (HFO-1123).

Aspect 6 is the method of any one of Aspects 1-5, wherein the catalyst is palladium.

Aspect 7 is the method of any one of Aspects 1-6, wherein the catalyst is supported on a support selected from the group consisting of carbon and alumina.

Aspect 8 is the method of any one of Aspects 1-7, wherein the catalyst is palladium supported on an alpha alumina support.

Aspect 9 is the method of any one of Aspects 1-8, wherein the catalyst is palladium supported on a theta alumina support.

Aspect 10 is the method of any one of Aspects 1-9, wherein the catalyst is palladium supported on a delta alumina support.

Aspect 11 is the method of any one of Aspects 1-10, wherein the product mixture comprises: about 50 mol. % to about 99.99 mol. % of 1,1,2-trifluoroethane (HFC-143); and about 0.01 mol. % to about 50 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the product mixture.

Aspect 12 is the method of Aspect 11, wherein the product mixture further comprises at least one of: 0.01 mol. % to 5 mol. % of ethane (HC-170), 0.01 mol. % to 5 mol. % of 1,1,1-trifluoroethane (HFC-143a), 0.01 mol. % to 15 mol. % of 1,1,1,2-tetrafluoroethane (HFC-134a), 0.01 mol. % to 4 mol. % of 1,1-difluoroethane (HFC-152a), 0.01 mol. % to 6 mol. % of one or more HCFC-142 isomers, and 0.01 mol. % to 2 mol. % of chloroethane (HCC-160), based on total moles of organic components in the product mixture.

Aspect 13 is the method of Aspect 12, wherein the one or more HCFC-142 isomers comprises 1-chloro-1,2-difluoroethane (HCFC-142a), 1-chloro-2,2-difluoroethane (HCFC-142), or 1-chloro-1,1-difluoroethane (HCFC-142b).

Aspect 14 is the method of any one of Aspects 1-13, wherein the reacting step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), and trifluoroethylene (HFO-1123) of greater than 70% and less than or equal to 100%.

Aspect 15 is the method of any one of Aspects 1-14, wherein the reacting step achieves a conversion of the reactant composition to 1,1,2-trifluoroethane (HFC-143) of greater than 80% and less than or equal to 100%.

Aspect 16 is the method of any one of Aspects 1-15, wherein the reacting step achieves a selectivity to ethane (HC-170), chloroethane (HCC-160), HCFC-142 isomers, and 1,1,1-trifluoroethane (HFC-143a) of less than 10% and greater than or equal to 0%.

Aspect 17 is 1,1,2-trifluoroethane (HFC-143), produced by the method of any one of Aspects 1-16.

Aspect 18 is the method of any one of Aspects 1-16, further comprising: dehydrohalogenating 1,1,2-trifluoroethane (HFC-143) in the presence of a catalyst to produce a composition comprising trans-1,2-difluoroethylene (HFO-1132E).

Aspect 19 is the method of Aspect 18, wherein the composition further comprises cis-1,2-difluoroethylene (HFO-1132Z).

Aspect 20 is the method of Aspect 19, further comprising: isomerizing cis-1,2-difluoroethylene (HFO-1132Z) to produce trans-1,2-difluoroethylene (HFO-1132E).

Aspect 21 is trans-1,2-difluoroethylene (HFO-1132E), produced by the method of Aspect 20.

Aspect 22 is the method of any one of Aspects 1-16, wherein the catalyst is selected from the group consisting of Fe, Co, and Ni; wherein loading of the catalyst on the support is from 10 wt. % to 80 wt. % based on the total weight of the catalyst and the support.

Aspect 23 is the method of any one of Aspects 1-16, wherein the catalyst is selected from the group consisting of Pd, Pt, Rh, Ru, and Ir; wherein loading of the catalyst on the support is from 0.01 wt. % to 10 wt. % based on the total weight of the catalyst and the support.

Aspect 24 is the method of any one of Aspects 1-16, wherein the catalyst is palladium supported on an alpha alumina support; wherein the temperature is between about 75° C. and about 150° C.; wherein the product mixture comprises: from 90 mol. % to 95 mol. % of 1,1,2-trifluoroethane (HFC-143); and from 5 mol. % to 10 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the product mixture; wherein loading of palladium on the alpha alumina support is from 0.1 wt. % to 0.5 wt. % based on the total weight of the catalyst and the support.

Aspect 25 is the method of Aspect 24, wherein the product mixture comprises from 85 mol. % to 95 mol. % of 1,1,2-trifluoroethane (HFC-143) and at least one of 1-chloro-1,1,2-trifluoroethane (HCFC-133b), 1-chloro-1,2,2-trifluoroethane (HCFC-133), and trifluoroethylene (HFO-1123), based on total moles of organic components in the product mixture.

Aspect 26 is the method of Aspect 25, wherein the product mixture further comprises at least one of: 0.01 mol. % to 1 mol. % of ethane (HC-170), 0.01 mol. % to 1.6 mol. % of 1,1,1-trifluoroethane (HFC-143a), 0.01 mol. % to 0.5 mol. % of 1,1,1,2-tetrafluoroethane (HFC-134a), 0.01 mol. % to 1.7 mol. % of 1,1-difluoroethane (HFC-152a), 0.01 mol. % to 0.15 mol. % of one or more HCFC-142 isomers, and 0.01 mol. % to 0.5 mol. % of chloroethane (HCC-160), based on total moles of organic components in the product mixture.

Aspect 27 is the method of any one of Aspects 1-16, wherein the catalyst is palladium supported on a theta alumina support; wherein the temperature is between about 100° C. and about 150° C.; wherein the product mixture comprises: from 85 mol. % to 90 mol. % of 1,1,2-trifluoroethane (HFC-143); and from 10 mol. % to 15 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the product mixture; wherein loading of palladium on the theta alumina support is from 0.1 wt. % to 0.5 wt. % based on the total weight of the catalyst and the support.

Aspect 28 is the method of Aspect 27, wherein the product mixture comprises from 90 mol. % to 98 mol. % of 1,1,2-trifluoroethane (HFC-143) and at least one of 1-chloro-1,1,2-trifluoroethane (HCFC-133b), 1-chloro-1,2,2-trifluoroethane (HCFC-133), and trifluoroethylene (HFO-1123), based on total moles of organic components in the product mixture.

Aspect 29 is the method of Aspect 28, wherein the product mixture further comprises at least one of: 0.01 mol. % to 0.5 mol. % of ethane (HC-170), 0.01 mol. % to 0.8 mol. % of 1,1,1-trifluoroethane (HFC-143a), 0.01 mol. % to 1 mol. % of 1,1,1,2-tetrafluoroethane (HFC-134a), 0.01 mol. % to 0.3 mol. % of 1,1-difluoroethane (HFC-152a), 0.01 mol. % to 0.6 mol. % of one or more HCFC-142 isomers, and 0.01 mol. % to 0.3 mol. % of chloroethane (HCC-160), based on total moles of organic components in the product mixture.

Aspect 30 is the method of any one of Aspects 1-16, wherein the catalyst is palladium supported on a delta alumina support; wherein the temperature is between about 100° C. and about 150° C.; wherein the product mixture comprises: from 60 mol. % to 65 mol. % of 1,1,2-trifluoroethane (HFC-143); and from 35 mol. % to 40 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the product mixture; wherein loading of palladium on the delta alumina support is from 0.1 wt. % to 0.5 wt. % based on the total weight of the catalyst and the support.

Aspect 31 is the method of Aspect 30, wherein the product mixture comprises from 98 mol. % to 99 mol. % of 1,1,2-trifluoroethane (HFC-143) and at least one of 1-chloro-1,1,2-trifluoroethane (HCFC-133b), 1-chloro-1,2,2-trifluoroethane (HCFC-133), and trifluoroethylene (HFO-1123), based on total moles of organic components in the product mixture.

Aspect 32 is the method of Aspect 31, wherein the product mixture further comprises at least one of: 0.01 mol. % to 0.1 mol. % of ethane (HC-170), 0.01 mol. % to 0.5 mol. % of 1,1,1-trifluoroethane (HFC-143a), 0.01 mol. % to 0.5 mol. % of 1,1,1,2-tetrafluoroethane (HFC-134a), 0.01 mol. % to 0.5 mol. % of 1,1-difluoroethane (HFC-152a), 0.01 mol. % to 0.1 mol. % of one or more HCFC-142 isomers, and 0.01 mol. % to 0.1 mol. % of chloroethane (HCC-160), based on total moles of organic components in the product mixture.

Aspect 33 is a composition comprising: from about 50 mol. % to about 99.99 mol. % of 1,1,2-trifluoroethane (HFC-143); and from about 0.01 mol. % to about 50 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the composition.

Aspect 34 is the composition of Aspect 33, further comprising at least one of: 0.01 mol. % to 5 mol. % of ethane (HC-170), 0.01 mol. % to 5 mol. % of 1,1,1-trifluoroethane (HFC-143a), 0.01 mol. % to 15 mol. % of 1,1,1,2-tetrafluoroethane (HFC-134a), 0.01 mol. % to 4 mol. % of 1,1-difluoroethane (HFC-152a), 0.01 mol. % to 6 mol. % of one or more HCFC-142 isomers, and 0.01 mol. % to 2 mol. % of chloroethane (HCC-160), based on total moles of organic components in the composition.

Aspect 35 is a composition comprising: from 90 mol. % to 95 mol. % of 1,1,2-trifluoroethane (HFC-143); and from 5 mol. % to 10 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the composition.

Aspect 36 is the composition of Aspect 35, further comprising at least one of: 0.01 mol. % to 1 mol. % of ethane (HC-170), 0.01 mol. % to 1.6 mol. % of 1,1,1-trifluoroethane (HFC-143a), 0.01 mol. % to 0.5 mol. % of 1,1,1,2-tetrafluoroethane (HFC-134a), 0.01 mol. % to 1.7 mol. % of 1,1-difluoroethane (HFC-152a), 0.01 mol. % to 0.15 mol. % of one or more HCFC-142 isomers, and 0.01 mol. % to 0.5 mol. % of chloroethane (HCC-160), based on total moles of organic components in the composition.

Aspect 37 is a composition comprising: from 85 mol. % to 90 mol. % of 1,1,2-trifluoroethane (HFC-143); and from 10 mol. % to 15 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the composition.

Aspect 38 is the composition of Aspect 37, further comprising at least one of: 0.01 mol. % to 0.5 mol. % of ethane (HC-170), 0.01 mol. % to 0.8 mol. % of 1,1,1-trifluoroethane (HFC-143a), 0.01 mol. % to 1 mol. % of 1,1,1,2-tetrafluoroethane (HFC-134a), 0.01 mol. % to 0.3 mol. % of 1,1-difluoroethane (HFC-152a), 0.01 mol. % to 0.6 mol. % of one or more HCFC-142 isomers, and 0.01 mol. % to 0.3 mol. % of chloroethane (HCC-160), based on total moles of organic components in the composition.

Aspect 39 is a composition comprising: from 60 mol. % to 65 mol. % of 1,1,2-trifluoroethane (HFC-143); and from 35 mol. % to 40 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the composition.

Aspect 40 is the composition of Aspect 39, further comprising at least one of: 0.01 mol. % to 0.1 mol. % of ethane (HC-170), 0.01 mol. % to 0.5 mol. % of 1,1,1-trifluoroethane (HFC-143a), 0.01 mol. % to 0.5 mol. % of 1,1,1,2-tetrafluoroethane (HFC-134a), 0.01 mol. % to 0.5 mol. % of 1,1-difluoroethane (HFC-152a), 0.01 mol. % to 0.1 mol. % of one or more HCFC-142 isomers, and 0.01 mol. % to 0.1 mol. % of chloroethane (HCC-160), based on total moles of organic components in the composition.

What is claimed is:

1. A method for producing 1,1,2-trifluoroethane (HFC-143), comprising:
reacting a reactant composition comprising chlorotrifluoroethylene (CTFE) with hydrogen in the presence of a catalyst at a temperature of between about 75° C. and about 225° C. to produce a product mixture comprising 1,1,2-trifluoroethane (HFC-143).

2. The method of claim 1, wherein the temperature is between about 100° C. and about 200° C.

3. The method of claim 1, wherein the temperature is between about 120° C. and about 180° C.

4. The method of claim 1, wherein the reactant composition further comprises trifluoroethylene (HFO-1123).

5. The method of claim 4, wherein the reactant composition comprises:
from about 50 mol. % to about 99.99 mol. % of chlorotrifluoroethylene (CTFE); and
from about 0.01 mol. % to about 50 mol. % of trifluoroethylene (HFO-1123), based on total moles of chlorotrifluoroethylene (CTFE) and trifluoroethylene (HFO-1123).

6. The method of claim 1, wherein the catalyst is palladium.

7. The method of claim 1, wherein the catalyst is supported on a support selected from the group consisting of carbon and alumina.

8. The method of claim 1, wherein the catalyst is palladium supported on an alpha alumina support.

9. The method of claim 1, wherein the catalyst is palladium supported on a theta alumina support.

10. The method of claim 1, wherein the catalyst is palladium supported on a delta alumina support.

11. The method of claim 1, wherein the product mixture comprises:
about 50 mol. % to about 99.99 mol. % of 1,1,2-trifluoroethane (HFC-143); and
about 0.01 mol. % to about 50 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the product mixture.

12. The method of claim 11, wherein the product mixture further comprises at least one of:
0.01 mol. % to 5 mol. % of ethane (HC-170),
0.01 mol. % to 5 mol. % of 1,1,1-trifluoroethane (HFC-143a),
0.01 mol. % to 15 mol. % of 1,1,1,2-tetrafluoroethane (HFC-134a),
0.01 mol. % to 4 mol. % of 1,1-difluoroethane (HFC-152a),
0.01 mol. % to 6 mol. % of one or more HCFC-142 isomers, and
0.01 mol. % to 2 mol. % of chloroethane (HCC-160), based on total moles of organic components in the product mixture.

13. The method of claim 12, wherein the one or more HCFC-142 isomers comprises 1-chloro-1,2-difluoroethane (HCFC-142a), 1-chloro-2,2-difluoroethane (HCFC-142), or 1-chloro-1,1-difluoroethane (HCFC-142b).

14. The method of claim 1, wherein the reacting step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), and trifluoroethylene (HFO-1123) of greater than 70% and less than or equal to 100%.

15. The method of claim 1, wherein the reacting step achieves a conversion of the reactant composition to 1,1,2-trifluoroethane (HFC-143) of greater than 80% and less than or equal to 100%.

16. The method of claim 1, wherein the reacting step achieves a selectivity to ethane (HC-170), chloroethane (HCC-160), HCFC-142 isomers, and 1,1,1-trifluoroethane (HFC-143a) of less than 10% and greater than or equal to 0%.

17. The method of claim 1, further comprising:
dehydrohalogenating 1,1,2-trifluoroethane (HFC-143) in the presence of a catalyst to produce a composition comprising trans-1,2-difluoroethylene (HFO-1132E) and cis-1,2-difluoroethylene (HFO-1132Z).

18. The method of claim 17, further comprising:
isomerizing cis-1,2-difluoroethylene (HFO-1132Z) to produce trans-1,2-difluoroethylene (HFO-1132E).

19. A composition comprising:
from 90 mol. % to 95 mol. % of 1,1,2-trifluoroethane (HFC-143); and
from 5 mol. % to 10 mol. % of at least one of 1-chloro-1,1,2-trifluoroethane (HCFC-133b), 1-chloro-1,2,2-trifluoroethane (HCFC-133), and trifluoroethylene (HFO-1123), based on total moles of HFC-143, HCFC-133b, HCFC-133, and HFO-1123 in the composition.

20. The composition of claim 19, further comprising at least one of:
0.01 mol. % to 1 mol. % of ethane (HC-170),
0.01 mol. % to 1.6 mol. % of 1,1,1-trifluoroethane (HFC-143a),
0.01 mol. % to 0.5 mol. % of 1,1,1,2-tetrafluoroethane (HFC-134a),
0.01 mol. % to 1.7 mol. % of 1,1-difluoroethane (HFC-152a),
0.01 mol. % to 0.15 mol. % of one or more HCFC-142 isomers, and
0.01 mol. % to 0.5 mol. % of chloroethane (HCC-160), based on total moles of organic components in the composition.
